(19) United States
(12) Patent Application Publication
(10) Pub. No.: US 2026/0297003 A1
(43) Pub. Date: Oct. 1, 2026

(54) METHODS OF CHEMICAL DECONSTRUCTION AND UPCYCLING OF POLYETHYLENE TEREPHTHALATE USING CARBONATE COMPOUNDS

(71) Applicant: UT-Battelle, LLC, Oak Ridge, TN (US)

(72) Inventors: Jeffrey C. Foster, Knoxville, TN (US); Nicholas J. Galan, Knoxville, TN (US); Tomonori Saito, Knoxville, TN (US)

(21) Appl. No.: 19/573,007

(22) Filed: Mar. 20, 2026

Related U.S. Application Data
(60) Provisional application No. 63/775,497, filed on Mar. 21, 2025.

(51) Int. Cl. C07C 37/50

(52) U.S. Cl. CPC C07C 37/50; C07C 2531/04

(57) ABSTRACT

A method of deconstructing and upcycling polyethylene terephthalate (PET) waste into at least one useful breakdown product by contacting the PET waste (1) with a catalyst that facilitates a transesterification reaction in the presence of an R-functionalized carbonate molecule (2) under transesterification conditions to produce a terephthalate monomer (3), according to the following reaction scheme:

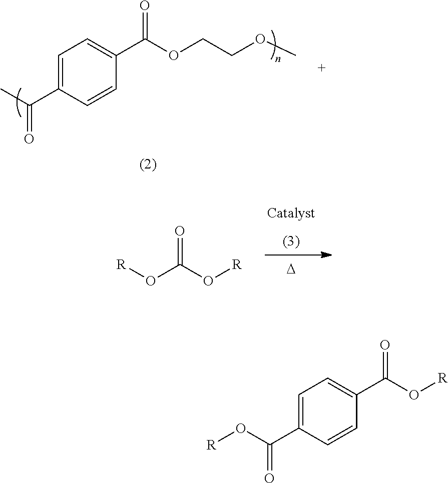

wherein: R is a hydrocarbon group containing 1-30 carbon atoms and optionally containing one or more heteroatoms; n is at least 10; and the symbol D denotes an elevated temperature sufficient to promote a transesterification reaction between (1) and (2) in the presence of the catalyst; provided that the R-functionalized carbonate molecule (2) functions as both a reactant and a solvent in the reaction, and no co-solvent is present in the reaction.

### CROSS REFERENCE TO RELATED APPLICATION

The present application claims benefit of U.S. Provisional Application No. 63/775,497, filed on Mar. 21, 2025, all of the contents of which are incorporated herein by reference.

### STATEMENT REGARDING FEDERALLY SPONSORED RESEARCH

This invention was made with government support under Prime Contract No. DE-AC05-000R22725 awarded by the U.S. Department of Energy. The government has certain rights in the invention.

### FIELD OF THE DISCLOSURE

The present disclosure generally relates to methods of breaking down and recycling plastic waste. The present disclosure more particularly relates to methods of deconstructing polyethylene terephthalate (PET) waste into useful products.

### BACKGROUND

Plastics are critical in facilitating the comfort and quality of everyday life. Most plastics are discarded after a single use, wasting the energy and carbon consumed for their production and incurring environmental costs. The production of commodity polymers in the U.S. alone requires an estimated 3.2 quadrillion BTUs of energy annually and generates 104 MMT CO2e of greenhouse gas (GHG) emissions. A 2018 study by the EPA revealed that 76% of produced plastic mass is landfilled annually, inevitably wasting the feedstock resources, energy, and carbon used for their production.

Thus, efficient closed-loop processes for plastics recycling and upcycling are needed to mitigate these losses and to combat the accumulation of plastic waste in the environment, which is now estimated to be on the order of 10⁸ tons of used, non-biodegradable material. Closed-loop production and recycling processes are needed to mitigate energy and carbon loss toward a net-zero carbon economy. Nevertheless, although much effort has been made in this respect, efficient and low cost processes for recycling and upcycling of plastic waste remains a significant challenge. PET is used in many commercial products, but only a fraction is mechanically recycled, and even less is chemically recycled. Efficient chemical recycling of PET represents an attractive solution, as small molecule terephthalates are used in myriad applications from plasticizers to resin crosslinkers, but conventional PET transesterification processes often suffer from equilibria issues associated with transesterification reactions particularly due to the vast excess of solvent used.

Commonly, PET deconstruction is achieved by an alcoholysis process in which a substantial excess of an alcohol solvent is used in conjunction with high temperatures, long reaction times and, in some cases, high pressures to achieve an acceptable yield of the desired deconstruction product. These non-ideal conditions can be attributed at least in part to the equilibrium issues associated with the release of reactive ethylene glycol (EG) during the deconstruction. Free EG can compete with alcohol employed for alcoholysis which often lead to the formation of byproducts such as bis-(2-hydroxyethyl) terephthalate. The conventional art attempts to avoid this by using a large excess of alcohol, long reaction times, and high temperatures to favor product formation, all of which make the conventional process inefficient and costly. Thus, there would be a significant benefit in a method that could achieve the deconstruction and upcycling of PET using substantially less solvent, shorter reaction times, and/or lower temperatures. Such a process would be further beneficial if it could maintain or even increase product selectivity and yield compared to the conventional process.

### SUMMARY

The present disclosure is directed to a novel method for deconstructing and upcycling polyethylene terephthalate (PET) waste into useful monomeric products. The method disclosed herein is advantageously cost efficient, straight-forward, and capable of being scaled up to make it industrially feasible. The method disclosed herein can advantageously achieve the deconstruction and upcycling of PET using substantially less solvent, shorter reaction times, and/or lower temperatures than conventional processes. The process described herein can also achieve an increased product selectivity and yield compared to the conventional process. The process can furthermore achieve all of this while producing substantially no ethylene glycol (EG) byproduct, in contrast to the conventional art which typically produces appreciable amounts of EG that lead to the formation of byproducts such as bis-(2-hydroxyethyl) terephthalate.

More particularly, the method includes the steps of: contacting the PET waste (1) with a catalyst that facilitates a transesterification reaction in the presence of an R-functionalized carbonate molecule (2) under transesterification conditions to produce a terephthalate monomer (3), according to the following reaction scheme:

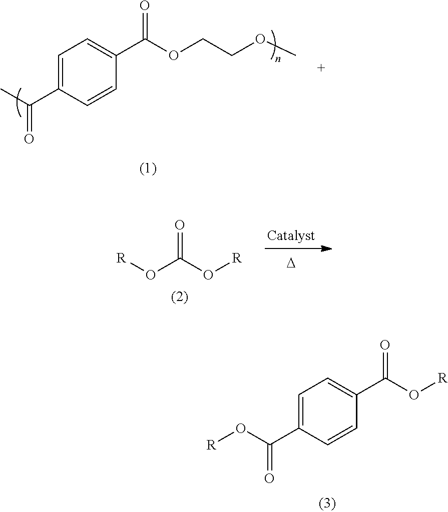

In the above scheme, R is a hydrocarbon group containing 1-30 carbon atoms and optionally containing one or more heteroatoms selected from N, O, S, P, B, Si, and halogen atoms, wherein the two R groups in molecule (2) may be the same or different and the two R groups in molecule (3) may be the same or different; n is at least 10; m is an integer from 1-12; and the symbol Δ denotes an elevated temperature sufficient to promote a transesterification reaction between (1) and (2) in the presence of the catalyst. Notably, in the reaction, the R-functionalized carbonate molecule (2) functions as both a reactant and a solvent in the reaction, and no co-solvent is present in the reaction. In particular embodiments, the catalyst comprises an organic nitrogen-containing base and a carboxylic acid or ester thereof. In some embodiments, the bicyclic guanidine is triazabicyclodecene (TBD) or 7-methyl-TBD. In some embodiments, the carboxylic acid is trifluoroacetic acid (TFA).

In some embodiments, the above described process produces a product (3) in which R is a hydrocarbon group containing 12-30 carbon atoms. In other embodiments, the above described process produces a product (3) in which R is a linear or branched alkyl or alkenyl group containing 1-30 carbon atoms. In separate or further embodiments, the above described process produces a product (3) in which R is a linear or branched alkyl or alkenyl group containing 12-30 carbon atoms. In separate or further embodiments, the above described process produces a product (3) in which R is a linear or branched alkyl group terminated by a polymerizable or reactive group. In separate or further embodiments, the above described process produces a product (3) in which one or both R groups is a heterocyclic ring or ring system (e.g., pyridine or thiophene), wherein the heterocyclic ring or ring system contains at least one ring heteroatom selected from N, O, and S, and the overall product (3) is non-polymeric.

This work achieves the production of a library of valuable organic molecules in high selectivity from waste PET. The present methodology is facilitated by symmetric or asymmetric hydrocarbyl (e.g., linear or branched alkyl or alkenyl) carbonates that can act as both a source of nucleophilic alcohol and as a by-product trap to shunt the reaction toward a single product with high selectivity. The carbonate compounds used in the presently described technology are environmentally friendly solvents that can be prepared from renewable sources. Moreover, the presently described deconstruction methodology can be achieved with fewer equivalents (e.g., 1-5) of the carbonate source to achieve full conversion to the desired product along with excellent selectivity. Further, the described method can facilitate the full deconstruction of PET within 1 hour or 30 minutes, which is significantly faster than conventional processes. Moreover, the terephthalate compounds being produced from waste PET are valuable and can be leveraged for a multitude of commercial applications.

The present disclosure is directed to a method for the selective deconstruction of PET to value-added terephthalate compounds using carbonates that serve multiple functions as follows: a reaction medium, an indirect source of nucleophile, and a kinetic sink for the evolving EG byproduct. This approach exploits the unique reactivity of carbonates, specifically their capacity to act as alkylating agents (via B_{AL}2 reactions) and their tendency to undergo rapid intramolecular cyclization reactions with EG to produce ethylene carbonate (EC). These factors combine to influence the transesterification equilibrium to suppress glycolated byproduct formation via the irreversible consumption of EG. Many of the advantages of the present methodology over PET alcoholysis technology is schematically shown in FIG. 1. The top reaction scheme in FIG. 1 shows PET deconstruction via alcoholysis, which, as shown, typically requires a vast excesses of alcohol, long reaction times, and produces glycolated byproducts. The bottom reaction scheme in FIG. 1 shows that PET deconstruction via the use of carbonates (as presently described) in place of alcohols significantly reduces solvent excess and reaction time, affording high isolated yields of valuable terephthalate products.

The methodology described herein demonstrates that carbonates in the absence of an alcohol or other co-solvent can be broadly employed for the deconstruction of PET in a manner that efficiently and selectively upcycles PET waste to produce a variety of valuable terephthalate compounds. Driving the deconstruction equilibrium towards CO₂ evolution using catalysts that promote EC polymerization enables efficient and selective polymer deconstruction with stochiometric or near-stoichiometric loadings of carbonate compounds. In fact, chemical deconstruction of PET using various carbonates was herein found to occur selectively with as little as one equivalent of carbonate, thus affording high isolated yields of functional terephthalate compounds. The present methodology has been found to be robust to impurities found in post-consumer polymeric material and could be deployed to deconstruct a variety of PET sources. Furthermore, the process provides an alternative route to methanolysis, with the capability of producing dimethylterephthalate (DMT) at much lower temperatures, pressures, and solvent volumes than the current state-of-the-art. Based on the high selectivity of the carbonate interchange deconstruction (CID) methodology described herein, along with the low cost and commercial availability of a broad variety of carbonates and their growing sustainability profile, the technologies described herein can provide a significant economic and environmental benefit. The technology described herein advantageously permits the utilization of waste condensation polymers as synthetic feedstocks while at the same time diminishing their accumulation in the environment.

Some additional capabilities of the presently described process are provided as follows:
1. Waste PET to monomer. Deconstruction of waste PET using dimethyl or diethyl carbonate provides dimethyl or diethyl terephthalate, respectively, which are common building blocks to produce PET.
2. Waste PET to resin crosslinking agents. Bis-epoxides and bis-alkenes (i.e., DAT) are commonly used as crosslinkers for the preparation of various thermosetting resins. Terminal alkenes can be leveraged in thiol-ene chemistries and olefin metathesis, for example, while epoxides can participate in a variety of ring-opening reactions with various nucleophiles, such as amines and carboxylic acids, to access polymer thermosets. Bis-alkenes can be prepared from CID of waste PET with carbonates bearing terminal alkenes while bis-epoxide bearing terephthalates can be prepared in two steps from waste PET (e.g., by reaction of PET with diallyl carbonate (DAC) to produce diallyl terephthalate (DAT), followed by reaction of DAT with mCPBA to produce diglycidyl terephthalate) in yields comparable to those seen in traditional methods.
3. Waste PET to plasticizers. Waste PET can be efficiently deconstructed with symmetric carbonates bearing hydrophobic carbon chains of various carbon lengths and branching architectures, thereby affording bis-alkyl terephthalates that are employed as commercial plasticizers. Alkyl chain lengths can vary from C₄-C₁₀ and include constitutional isomers with unique branching arrangements.
4. Waste PET to phase change materials. Waste PET can be efficiently deconstructed with symmetric carbonates bearing linear hydrophobic carbon chains of various lengths that promote crystallization, thereby affording bis-alkyl terephthalates that have shown promise for use as phase change materials in thermal energy storage applications.

As the terephthalate products described above are conventionally made from petroleum, the presently described alternative process provides an effective, highly energy efficient, and economical way to produce these building blocks from plastic waste. Moreover, the described process can find use in a number of industrial sectors, such as chemical supply companies, PET waste producers, resin producers, and end users for chemicals (e.g., resins).

The technologies described herein provide a platform for the production of various high value terephthalate molecules from waste PET deconstruction via carbonate interchange deconstruction reaction (FIG. 1). Numerous commercially available functional symmetric or asymmetric carbonates can be used as a carbonate source, including those that contain aromatic groups, alkyl ethers, alkynes, branched alkanes, and even thiophenes. Some examples are shown below, including those employing more unusual types of carbonates (e.g., those with heterocyclic rings). Any of these types of carbonates can be employed to afford high value terephthalates.

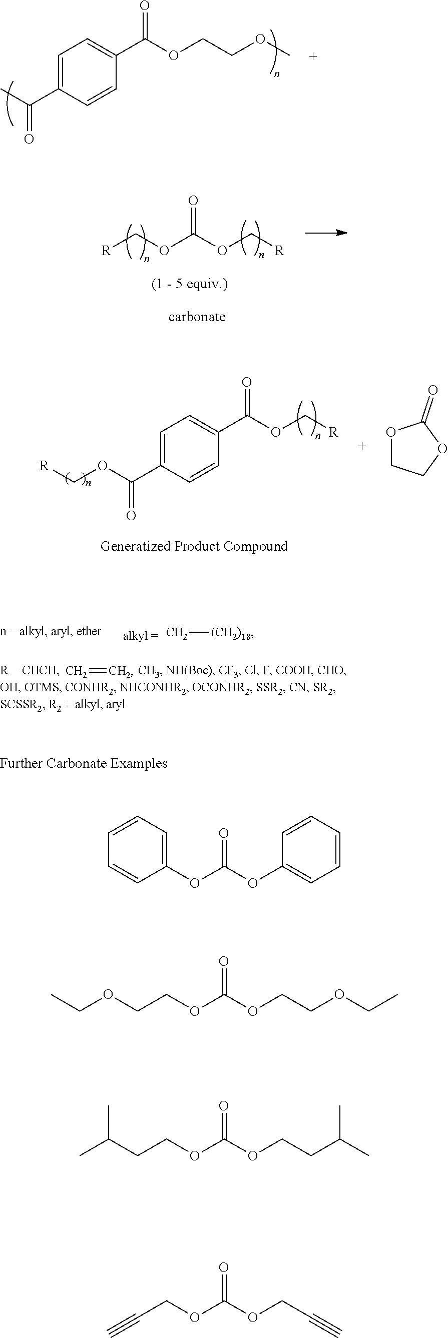

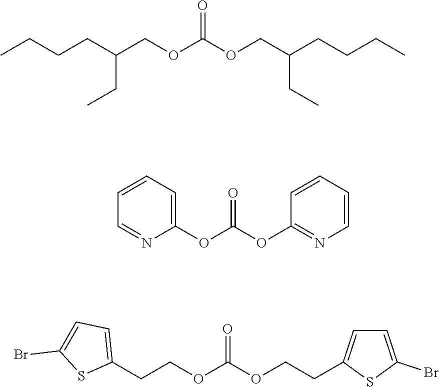 [SMILES: CCCCC(CC)COC(=O)OCC(CC)CCCC ; O=C(Oc1ccccn1)Oc1ccccn1 ; O=C(OCCc1ccc(Br)s1)OCCc1ccc(Br)s1]

### BRIEF DESCRIPTION OF THE FIGURES

FIG. 1. Top scheme: PET deconstruction via alcoholysis currently requires substantial excesses of alcohol and produces glycolated byproducts. Bottom scheme: The use of carbonates in place of alcohols for PET deconstruction significantly reduces solvent excess, affording high isolated yields of valuable terephthalate products.

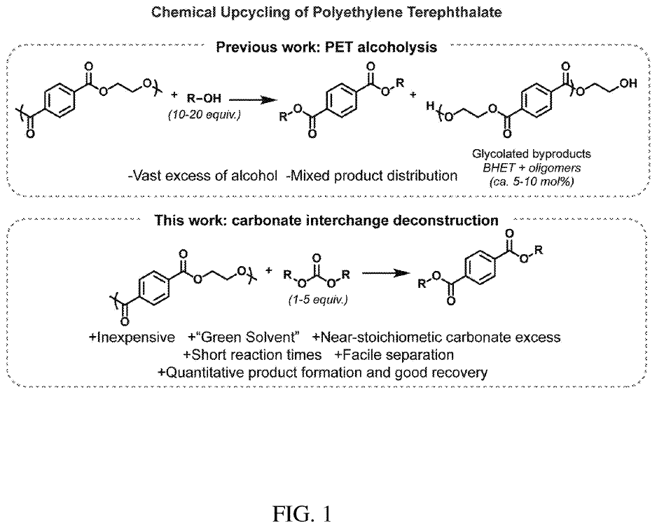

FIGS. 2A-2C. FIG. 2A is schematic depicting the preparation of difunctional terephthalates using alcohols or carbonates as the reaction medium and source of nucleophile. FIG. 2B is a plot of transesterification selectivity as a function of PET concentration for the various alcohols (orange closed symbols) and carbonates (blue open symbols). The solid lines represent concatenated linear fits of the alcohol and carbonate data. FIG. 2C is a graph showing a preliminary technoeconomic analysis derived from 0.5 g scale PET deconstructions using either 1-octanol or DOC as the solvent.

FIGS. 3A-3D. FIG. 3A schematically shows highlighted key reaction pathways during CID. DFT computed activation energies are shown for the steps of the transcarbonylation pathways involving carbonate. DMC was used as the carbonate for these calculations. FIG. 3B is a graph showing kinetic monitoring of PET deconstruction in the presence of 5 molar equiv. of DOC and 10 mol % TBD at 180° C. Conversion values were calculated from gravimetric analysis or 1H NMR spectroscopy. FIG. 3C photographically displays the qualitative determination of CO₂ formation using a pH indicator or by precipitation of CaCO₃. FIG. 3D is a graph showing the quantitative determination of CO₂ formed during CID via sampling of the reaction headspace followed by MS analysis to determine the μmol of CO₂ produced.

FIGS. 4A-4B. FIG. 4A shows high value terephthalate products accessible via CID. Reaction conversions and isolated yields are shown below each synthesized compound. FIG. 4B shows the synthesis of diglycidyl terephthalate (DGT) in two steps starting from PET egg carton waste.

### DETAILED DESCRIPTION

As used herein, the term “hydrocarbon group” (also denoted by the group R) is defined as a chemical group composed of at least of carbon and hydrogen. In some embodiments, the hydrocarbon group is composed solely of carbon and hydrogen. In other embodiments, the hydrocarbon group may (i.e., optionally) be substituted with one or more fluorine atoms to result in partial or complete fluorination of the hydrocarbon group. In other embodiments, as further discussed below, the hydrocarbon group may be substituted with one or more other heteroatoms (e.g., N, O, S, P, B, and/or Si) or heteroatom-containing groups containing any one or more of these heteroatoms.

The hydrocarbon group (R) typically contains 1-30 carbon atoms. In different embodiments, one or more of the hydrocarbon groups may contain, for example, 1, 2, 3, 4, 5, 6, 7, 8, 9, 10, 12, 15, 18, 20, 22, 24, 26, 28, or 30 carbon atoms, or a number of carbon atoms within a particular range bounded by any two of the foregoing carbon numbers (e.g., 1-80, 1-50, 3-80, 3-50, 1-30, 2-30, 3-30, 4-30, 6-30, 8-30, 10-30, 12-30, 1-20, 6-20, 8-20, 10-20, or 12-20 carbon atoms). Hydrocarbon groups in different compounds described herein, or in different positions of a compound, may possess the same or different number (or preferred range thereof) of carbon atoms in order to independently adjust or optimize such properties as the complexing ability, extracting (extraction affinity) ability, or selectivity ability.

In a first set of embodiments, the hydrocarbon group (R) is a saturated and straight-chained group, i.e., a straight-chained (linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-eicosyl, n-docosyl, n-tetracosyl, n-hexacosyl, n-octacosyl, and n-triacontyl groups.

In a second set of embodiments, the hydrocarbon group (R) is saturated and branched, i.e., a branched alkyl group. Some examples of branched alkyl groups include isopropyl (2-propyl), isobutyl(2-methylprop-1-yl), sec-butyl(2-butyl), t-butyl (1,1-dimethylethyl-1-yl), 2-pentyl, 3-pentyl, 2-methylbut-1-yl, isopentyl(3-methylbut-1-yl), 1,2-dimethylprop-1-yl, 1,1-dimethylprop-1-yl, neopentyl(2,2-dimethylprop-1-yl), 2-hexyl, 3-hexyl, 2-methylpent-1-yl, 3-methylpent-1-yl, isohexyl(4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, 1,2,2-trimethylprop-1-yl, isoheptyl, isooctyl, and the numerous other branched alkyl groups having up to 20 or 30 carbon atoms, wherein the “1-yl” suffix represents the point of attachment of the group.

In a third set of embodiments, the hydrocarbon group (R) is saturated and cyclic, i.e., a cycloalkyl group. Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

In a fourth set of embodiments, the hydrocarbon group (R) is unsaturated and straight-chained, i.e., a straight-chained (linear) olefinic or alkenyl group. The unsaturation occurs by the presence of one or more carbon-carbon double bonds and/or one or more carbon-carbon triple bonds. Some examples of straight-chained olefinic groups include vinyl, propen-1-yl(allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl (2-propynyl), 3-butynyl, and the numerous other straight-chained alkenyl or alkynyl groups having up to 20 or 30 carbon atoms.

In a fifth set of embodiments, the hydrocarbon group (R) is unsaturated and branched, i.e., a branched olefinic or alkenyl group. Some examples of branched olefinic groups include propen-2-yl (CH₂═C.—CH₃), 1-buten-2-yl (CH₂═C.—CH₂—CH₃), 1-buten-3-yl (CH₂═CH—CH.—CH₃), 1-propen-2-methyl-3-yl (CH₂═C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, and the numerous other branched alkenyl groups having up to 20 or 30 carbon atoms, wherein the dot in any of the foregoing groups indicates a point of attachment.

In a sixth set of embodiments, the hydrocarbon group (R) is unsaturated and cyclic, i.e., a cycloalkenyl group. The unsaturated cyclic group may be aromatic or aliphatic. Some examples of unsaturated cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group may or may not also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene fused ring systems.

As indicated earlier above, any of the hydrocarbon groups described above may be substituted with one or more fluorine atoms. As an example, an n-octyl group may be substituted with a single fluorine atom to result in, for example, a 7-fluorooctyl or 8-fluorooctyl group, or substituted with two or more fluorine atoms to result in, for example, 7,8-difluorooctyl, 8,8-difluorooctyl, 8,8,8-trifluorooctyl, or perfluorooctyl group. Any of the hydrocarbon groups described herein may contain a single ether (—O—) or thioether (—S—) linkage connecting between carbon atoms in the hydrocarbon group. An example of a hydrocarbon group containing a single ether or thioether group is —(CH₂)₂—X—(CH₂)₇CH₃, wherein X represents O or S.

Any of the hydrocarbon groups described above may (or may not) be substituted with one or more heteroatom-containing groups. Some examples of heteroatom-containing groups include —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂ groups, wherein R″ groups are independently selected from H and alkyl groups containing 1-12 carbon atoms. In some embodiments, an R group contains one or more hydroxy-containing groups, ether-containing groups, or one or more groups containing both hydroxy and ether groups. The groups may also contain two or more hydroxy or ether groups, thereby corresponding to polyhydroxy or polyether groups, respectively. Some examples of hydroxy-containing groups include —CH₂CH₂OH, —CH(OH)CH₂(OH), —CH(OH)CH₂(OH), —CH[CH₂(OH)]₂, and —CH(OH)CH(OH)CH₂(OH) groups. Some examples of ether-containing groups include —CH₂OCH₃, —CH₂CH₂OCH₃, —CH₂OCH₂CH₃, —CH₂CH₂OCH₂CH₃, —CH₂OCH₂CH₂OCH₃, —CH₂OCH₂CH₂OCH₂CH₃, —CH[CH₂(OCH₃)]₂, —CH[CH₂(OCH₂CH₂OCH₃)]₂, —(CH₂CH₂O)ₘCH₂CH₃, —CH₂CH₂O)ₘH, —CH₂O(CH₂CH₂O)ₘH, and —CH₂O(CH₂CH₂O)ₘCH₃, where m is at least 1, 2, 3, 4, 5, or 6. Some examples of hydrophilic groups containing both ether and hydroxy groups include —(CH₂CH₂O)ₚ—CH₂CH₂OH, wherein p is 0, 1, 2, 3, 4, 5, and 6 or within a range therein (e.g., 0-6, 1-6, or 2-6).

In embodiments where the hydrocarbon group (R) is or includes a cyclic group, the cyclic group may be monocyclic by containing a single ring without connection or fusion to another ring. The cyclic hydrocarbon group may alternatively be, for example, bicyclic, tricyclic, tetracyclic, or a higher polycyclic ring system by having at least two rings interconnected (i.e., by a bond) and/or fused.

In some embodiments, the cyclic hydrocarbon group is carbocyclic, i.e., does not contain ring heteroatoms (i.e., only ring carbon atoms). In different embodiments, ring carbon atoms in the carbocyclic group are all saturated, or a portion of the ring carbon atoms are unsaturated, or the ring carbon atoms are all unsaturated, as found in aromatic carbocyclic groups, which may be monocyclic, bicyclic, tricyclic, or higher polycyclic aromatic groups.

In some embodiments, the hydrocarbon group (R) is or includes a cyclic or polycyclic group that includes at least one ring heteroatom (for example, one, two, three, four, or higher number of heteroatoms). Such ring heteroatom-substituted cyclic groups are referred to herein as “heterocyclic groups”. As used herein, a “ring heteroatom” is an atom other than carbon and hydrogen (typically, selected from nitrogen, oxygen, and sulfur) that is inserted into, or replaces a ring carbon atom in a hydrocarbon ring structure. In some embodiments, the heterocyclic group is saturated, while in other embodiments, the heterocyclic group is unsaturated (i.e., aliphatic or aromatic heterocyclic groups, wherein the aromatic heterocyclic group is also referred to herein as a “heteroaromatic ring” or a “heteroaromatic fused-ring system” in the case of at least two fused rings, at least one of which contains at least one ring heteroatom).

Some examples of saturated heterocyclic groups (R) containing at least one oxygen atom include oxetane, tetrahydrofuran, tetrahydropyran, 1,4-dioxane, 1,3-dioxane, and 1,3-dioxepane rings. Some examples of saturated heterocyclic groups (R) containing at least one nitrogen atom include pyrrolidine, piperidine, piperazine, imidazolidine, azepane, and decahydroquinoline rings. Some examples of saturated heterocyclic groups (R) containing at least one sulfur atom include tetrahydrothiophene, tetrahydrothiopyran, 1,4-dithiane, 1,3-dithiane, and 1,3-dithiolane rings. Some examples of saturated heterocyclic groups (R) containing at least one oxygen atom and at least one nitrogen atom include morpholine and oxazolidine rings. An example of a saturated heterocyclic group containing at least one oxygen atom and at least one sulfur atom includes 1,4-thioxane. An example of a saturated heterocyclic group containing at least one nitrogen atom and at least one sulfur atom includes thiazolidine and thiamorpholine rings.

Some examples of unsaturated heterocyclic groups (R) containing at least one oxygen atom include furan, pyran, 1,4-dioxin, benzofuran, dibenzofuran, and dibenzodioxin rings. Some examples of unsaturated heterocyclic groups (R) containing at least one nitrogen atom include pyrrole, imidazole, pyrazole, pyridine, pyrazine, pyrimidine, 1,3,5-triazine, azepine, diazepine, indole, purine, benzimidazole, indazole, 2,2′-bipyridine, quinoline, isoquinoline, phenanthroline, 1,4,5,6-tetrahydropyrimidine, 1,2,3,6-tetrahydropyridine, 1,2,3,4-tetrahydroquinoline, quinoxaline, quinazoline, pyridazine, cinnoline, 5,6,7,8-tetrahydroquinoxaline, 1,8-naphthyridine, and 4-azabenzimidazole rings. Some examples of unsaturated heterocyclic groups containing at least one sulfur atom include thiophene, thianaphthene, and benzothiophene rings. Some examples of unsaturated heterocyclic groups (R) containing at least one oxygen atom and at least one nitrogen atom include oxazole, isoxazole, benzoxazole, benzisoxazole, oxazoline, 1,2,5-oxadiazole (furazan), and 1,3,4-oxadiazole rings. Some examples of unsaturated heterocyclic groups (R) containing at least one nitrogen atom and at least one sulfur atom include thiazole, isothiazole, benzothiazole, benzoisothiazole, thiazoline, and 1,3,4-thiadiazole rings.

The present disclosure is foremost directed to a method of deconstructing and upcycling polyethylene terephthalate (PET) waste into at least one useful monomeric product. The PET waste can contain only PET, or the PET waste may refer to a mixed plastic waste containing PET. Some examples of waste plastics other than PET that may be included in a mixed plastic waste stream include one or more of, for example, polypropylene (PP), polyethylene (PE), polyurethane (PU), polycarbonate (PC), polystyrene (PS), and/or polyamide (PA). The present invention can successfully deconstruct and upcycle PET in a mixed plastic waste stream that contains one or more of any of the above exemplary plastics other than PET.

The method is conducted under transesterification conditions, which include a temperature range and possibly other conditions that promote transesterification between PET and a carbonate molecule. In the method, PET waste (1) is contacted with a catalyst that facilitates a transesterification reaction in the presence of an R-functionalized carbonate molecule (2) under transesterification conditions to produce a terephthalate monomer (3), according to the following reaction scheme:

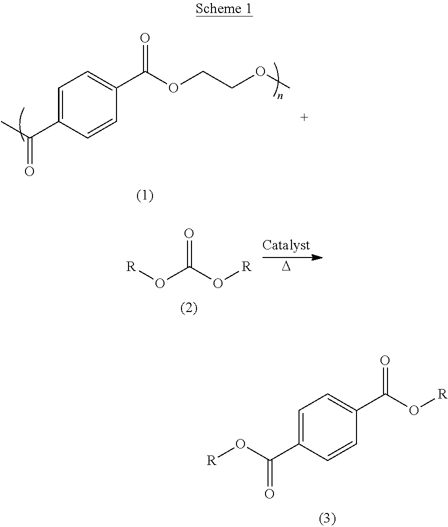

The variable R in the above Scheme 1 is a hydrocarbon group containing 1-30 carbon atoms and optionally containing one or more heteroatoms selected from N, O, S, P, B, Si, and halogen atoms, as described above, wherein the two R groups in molecule (2) may be the same or different and the two R groups in molecule (3) may be the same or different. In a specific embodiment, R is a hydrocarbon group (or more specifically, a linear or branched alkyl or alkenyl group) containing 1-30 carbon atoms, or more specifically, 1-12 carbon atoms or 12-30 carbon atoms. In another specific embodiment, R is a linear or branched alkyl group terminated by a polymerizable or reactive group and may contain any of the foregoing number of carbon atoms. In another specific embodiment, at least one R is a heterocyclic ring or ring system, such as any of the generic or specific types described above, or more particularly wherein the heterocyclic ring or ring system contains at least one ring heteroatom selected from N, O, and S, and the overall compound is non-polymeric. The variable n is at least 10, 20, 30, 40, 50, or 100.

The carbonate molecule (2) can be obtained commercially or can be synthesized by means well known in the art. For example, dioctyl carbonate can be produced by the transesterification of dimethyl carbonate with n-octanol. Alternatively, an alcohol (e.g., n-butanol, n-octanol, n-decanol, 2-hydroxypyridine or 2-hydroxythiophene) may be reacted with phosgene to form a carbonate molecule functionalized with butyl, octyl, decyl, pyridine, or thiophene groups, respectively; or 2-(hydroxyethyl)pyridine or 2-(hydroxyethyl)thiophene may be reacted with phosgene to form a carbonate molecule containing pyridine or thiophene attached to the carbonate moiety through an ethyl linker.

The symbol Δ in the above Scheme 1 denotes an elevated temperature sufficient to promote a transesterification reaction between (1) and (2) in the presence of the catalyst to produce the monomer (3). The elevated temperature is typically at least 50° C. and up to 200° C., provided that the temperature is below the decomposition temperature of the PET waste. In different embodiments, the temperature may be, for example, 50° C., 60° C., 70° C., 80° C., 90° C., 100° C., 110° C., 120° C., 130° C., 140° C., 150° C., 160° C., 170° C., 180° C., 190° C., or 200° C., or a temperature within a range bounded by any two of the foregoing values (e.g., 50-200° C., 50-180° C., 50-150° C., 50-120° C., 50-100° C., 70-200° C., 70-180° C., 70-150° C., 70-120° C., or 70-100° C.). The process may be conducted at any of the foregoing temperatures for a period of time of, for example, 0.1 hour, 0.2 hour, 0.3 hour, 0.4 hour, 0.5 hour, 1 hour, 2 hours, 3 hours, 4 hours, 5 hours, or 6 hours, or a period of time within a range therein (e.g., 0.1-6 hours, 0.3-6 hours, 0.5-6 hours, 1-6 hours, 0.1-4 hours, 0.3-4 hours, 0.5-4 hours, 1-4 hours, 0.1-3 hours, 0.3-3 hours, 0.5-3 hours, 1-3 hours, 0.1-2 hours, 0.3-2 hours, 0.5-2 hours, 1-2 hours, 0.1-1 hours, 0.3-1 hours, 0.5-1 hours, or 0.1-0.5 hours. The method may be conducted at ambient pressure (about 1 atm) but may, in some embodiments, subject the combined components (polymer waste in contact with catalyst and carbonate) to a reduced pressure (e.g., less than 1 or 0.1 atm) or an elevated pressure, e.g., above 1 atm, or at least 2, 5, 10, 20, or 50 atm. In the method, the PET waste (1) is completely converted to terephthalate monomer (3) within any of the periods of time provided above, e.g., no more than 6 hours, 5 hours, 4 hours, 3 hours, 2 hours, or 1 hour, or within any of the time ranges provided above.

The catalyst used in the method of upcycling PET facilitates a transesterification reaction in the presence of an R-functionalized carbonate molecule (2) under transesterification conditions to produce a terephthalate monomer (3), according to the reaction scheme provided above. Some examples of catalysts that have this ability include organic nitrogen-containing bases (or more specifically, guanidine or superbases), dimethylaminopyridine (DMAP), ionic liquids (or more specifically, imidazolium ionic liquids), potassium t-butoxide (KOtBu), N,N-Diisopropylethylamin (DIPEA), potassium carbonate (K₂CO₃), zinc acetate (Zn(OAc)₂), p-toluenesulfonic acid (TsOH), and sulfuric acid (H₂SO₄).

In some embodiments, the catalyst used in the method of upcycling PET is an organic nitrogen-containing base (i.e., “base” or “B species”) or a combination of such a base or B species and a carboxylic acid or ester thereof (i.e., “acid” or “A species”). The base is typically complexed with the acid in the catalyst. The carboxylic acid or ester (A) species may be combined with the base (B) species in any desired B:A molar ratio, wherein the B:A molar ratio may have a B molar value and A molar value independently selected from, for example, 0.05, 0.06, 0.07, 0.08, 0.09, 0.1, 0.2, 0.25, 0.3, 0.4, 0.5, 0.6, 0.7, 0.8, 0.9, and 1. The B:A molar ratio may also be within a range of B:A molar ratios, such as a range of 0.05:1 to 1:0.05 (1:20 to 20:1). In other embodiments, the B:A molar ratio is within a range of 0.2:1 to 1:0.2 (or 1:5 to 5:1). In other embodiments, the B:A molar ratio is within a range of 0.5:1 to 1:0.5 (or 1:2 to 2:1). In other embodiments, the B:A molar ratio is about 1:1.

In embodiments, the organic nitrogen-containing base has the following structure:

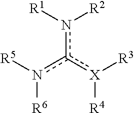

In Formula (1), the variables R¹, R², R³, R⁴, R⁵, and R⁶ are independently selected from hydrogen atom, electron pair, and alkyl groups containing one to three carbon atoms. Examples of alkyl groups containing one to three carbon atoms include methyl, ethyl, n-propyl, and isopropyl. The dotted lines represent optional double bonds. If a nitrogen atom in Formula (1) or sub-formula thereof is engaged in a carbon-nitrogen double bond, a substituent shown attached to the nitrogen atom is an electron pair to maintain charge neutrality. The variable X is C, CR^{a}, or N, wherein R^{a} is selected from hydrogen atom and alkyl groups containing one to three carbon atoms. Notably, by the rules of chemistry, the variable R¹, R², R³, R⁴, R⁵, or R⁶ can only be an electron pair when the electron pair is attached to a nitrogen atom engaged in a carbon-nitrogen double bond. Similarly, by the rules of chemistry, X is C only when C is engaged in a carbon-carbon double bond, and X is CR^{a} only when the carbon atom in CR^{a} is engaged exclusively in carbon-carbon single bonds. In some embodiments, all of R¹, R², R³, R⁴, R⁵, and R⁶ are hydrogen atoms. In other embodiments, one, two, three, four, five, or all of R¹, R², R³, R⁴, R⁵, and R⁶ are alkyl groups containing one to three carbon atoms, or more particularly, one, two, three, four, five, or all of R¹, R², R³, R⁴, R⁵, and R⁶ are selected from methyl groups, ethyl groups, n-propyl groups, or isopropyl groups or selected from a combination thereof.

In a first set of embodiments, R¹ and R² optionally interconnect to form a five, six, or seven-membered ring. In some embodiments, R¹ and R² interconnect to form a five, six, or seven-membered ring, while in other embodiments, R¹ and R² do not interconnect.

In a second set of embodiments, R² and R³ optionally interconnect to form a five, six, or seven-membered ring. In some embodiments, R² and R³ interconnect to form a five, six, or seven-membered ring, while in other embodiments, R² and R³ do not interconnect.

In a third set of embodiments, R³ and R⁴ optionally interconnect to form a five, six, or seven-membered ring. In some embodiments, R³ and R⁴ interconnect to form a five, six, or seven-membered ring, while in other embodiments, R³ and R⁴ do not interconnect.

In a fourth set of embodiments, R⁴ and R⁶ optionally interconnect to form a five, six, or seven-membered ring. In some embodiments, R⁴ and R⁶ interconnect to form a five, six, or seven-membered ring, while in other embodiments, R⁴ and R⁶ do not interconnect.

In a fourth set of embodiments, R⁵ and R⁶ optionally interconnect to form a five, six, or seven-membered ring. In some embodiments, R⁵ and R⁶ interconnect to form a five, six, or seven-membered ring, while in other embodiments, R⁵ and R⁶ do not interconnect.

In a fifth set of embodiments, R¹ and R⁵ optionally interconnect to form a five, six, or seven-membered ring. In some embodiments, R¹ and R⁵ interconnect to form a five, six, or seven-membered ring, while in other embodiments, R¹ and R⁵ do not interconnect.

Any two of the above first to fifth embodiments may be combined to result in a bicyclic compound within the scope of Formula (1). Any three of the above first to fifth embodiments may be combined to result in a tricyclic compound within the scope of Formula (1).

In some embodiments of Formula (1), the organic nitrogen-containing base has the following structure:

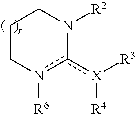

In Formula (1a), R², R³, R⁴, R⁶ and X are as defined earlier above, and the variable r is a value of 0, 1, or 2. When r is 0, a five-membered ring results. When r is 1, a six-membered ring results. When r is 2, a seven-membered ring results. In some embodiments of Formula (1a), X is C or CR^{a}. In other embodiments of Formula (1a), X is N. In some embodiments, all of R², R³, R⁴, R⁶ are hydrogen atoms. In other embodiments, one, two, three, or all of R², R³, R⁴, R⁶ are alkyl groups containing one to three carbon atoms, or more particularly, one, two, three, or all of R², R³, R⁴, R⁶ are selected from methyl groups, ethyl groups, n-propyl groups, or isopropyl groups or selected from a combination thereof. In the case where a nitrogen atom in Formula (1a) is attached to a hydrogen atom or alkyl group, the nitrogen atom is not engaged in a double bond since the presence of a double bond in such circumstance would result in a positively charged molecule, but molecules of Formula (1) and sub-formulas thereof are uncharged (neutral) for purposes of the present disclosure. If a nitrogen atom in Formula (1) or sub-formula thereof is engaged in a carbon-nitrogen double bond, the substituent (e.g., R⁴ or R⁶) is an electron pair in that case to maintain charge neutrality (no charge). In some embodiments, R⁶ is an electron pair and the nitrogen atom attached to R⁶ is engaged in a carbon-nitrogen double bond, in which case the remaining optional double bond shown in Formula (1a) is not present. In further or alternative embodiments, R⁴ is an alkyl group, such as any of the alkyl groups provided above, particularly methyl or ethyl. In further or alternative embodiments, R² and R³ optionally interconnect to form a five, six, or seven-membered ring. In further or alternative embodiments, R³ and R⁴ optionally interconnect to form a five, six, or seven-membered ring. In further or alternative embodiments, R⁴ and R⁶ optionally interconnect to form a five, six, or seven-membered ring.

In some embodiments of Formula (1a), the organic nitrogen-containing base has the following structure:

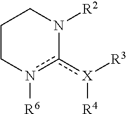

In Formula (1b), R², R³, R⁴, R⁶ and X are as defined earlier above. In some embodiments of Formula (1b), X is C or CR^{a}. In other embodiments of Formula (1b), X is N. In some embodiments, all of R², R³, R⁴, R⁶ are hydrogen atoms. In other embodiments, one, two, three, or all of R², R³, R⁴, R⁶ are alkyl groups containing one to three carbon atoms, or more particularly, one, two, three, or all of R², R³, R⁴, R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1a) is not present. In further or alternative embodiments, R⁴ is an alkyl group, such as any of the alkyl groups provided above, particularly methyl or ethyl. In further or alternative embodiments, R² and R³ optionally interconnect to form a five, six, or seven-membered ring. In further or alternative embodiments, R³ and R⁴ optionally interconnect to form a five, six, or seven-membered ring. In further or alternative embodiments, R⁴ and R⁶ optionally interconnect to form a five, six, or seven-membered ring.

In some embodiments of Formula (1), the organic nitrogen-containing base has the following structure:

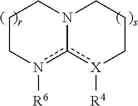

In Formula (1c), R⁴, R⁶ and X are as defined earlier above, and the variables r and s are each independently a value of 0, 1, or 2. When r or s is 0, a five-membered ring results. When r or s is 1, a six-membered ring results. When r or s is 2, a seven-membered ring results. In different embodiments, r and s are both 0; or r and s are both 1; or r and s are both 2; or r is 0 and s is 1; or r is 0 and s is 2; orris 1 and s is 0; orris 1 and s is 2; or r is 2 and s is 0; or r is 2 and s is 1. In some embodiments of Formula (1c), X is C or CR^{a}. In other embodiments of Formula (1c), X is N. In some embodiments, R⁴ and R⁶ are hydrogen atoms. In other embodiments, one or both of R⁴ and R⁶ are alkyl groups containing one to three carbon atoms, or more particularly, one or both of R⁴ and R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1c) is not present. In further or alternative embodiments, R⁴ is an alkyl group, such as any of the alkyl groups provided above, particularly methyl or ethyl. In further or alternative embodiments, R⁴ and R⁶ optionally interconnect to form a five, six, or seven-membered ring.

In some embodiments of Formula (1c), the organic nitrogen-containing base has the following structure:

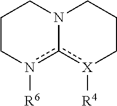

In Formula (1d), R⁴, R⁶ and X are as defined earlier above. In some embodiments of Formula (1d), X is C or CR^{a}. In other embodiments of Formula (1d), X is N. In some embodiments, R⁴ and R⁶ are hydrogen atoms. In other embodiments, one or both of R⁴ and R⁶ are alkyl groups containing one to three carbon atoms, or more particularly, one or both of R⁴ and R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1d) is not present. In further or alternative embodiments, R⁴ is an alkyl group, such as any of the alkyl groups provided above, particularly methyl or ethyl. In further or alternative embodiments, R⁴ and R⁶ optionally interconnect to form a five, six, or seven-membered ring.

In some embodiments of Formula (1c), the organic nitrogen-containing base has the following structure:

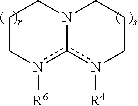

In Formula (1e), R⁴ and R⁶ are as defined earlier above, and the variables r and s are each independently a value of 0, 1, or 2. When r or s is 0, a five-membered ring results. When r or s is 1, a six-membered ring results. When r or s is 2, a seven-membered ring results. In different embodiments, r and s are both 0; or r and s are both 1; or r and s are both 2; or r is 0 and s is 1; or r is 0 and s is 2; orris 1 and s is 0; orris 1 and s is 2; or r is 2 and s is 0; or r is 2 and s is 1. In some embodiments, R⁴ and R⁶ are hydrogen atoms. In other embodiments, one or both of R⁴ and R⁶ are alkyl groups containing one to three carbon atoms, or more particularly, one or both of R⁴ and R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1e) is not present. In further or alternative embodiments, R⁴ is an alkyl group, such as any of the alkyl groups provided above, particularly methyl or ethyl. In further or alternative embodiments, R⁴ and R⁶ optionally interconnect to form a five, six, or seven-membered ring.

In some embodiments of Formula (1e), the organic nitrogen-containing base has the following structure:

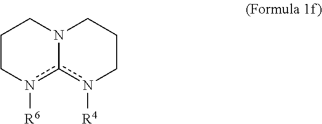

In Formula (1f), R⁴ and R⁶ are as defined earlier above. In some embodiments, R⁴ and R⁶ are hydrogen atoms. In other embodiments, one or both of R⁴ and R⁶ are alkyl groups containing one to three carbon atoms, or more particularly, one or both of R⁴ and R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1f) is not present. In further or alternative embodiments, R⁴ is an alkyl group, such as any of the alkyl groups provided above, particularly methyl or ethyl. In further or alternative embodiments, R⁴ and R⁶ optionally interconnect to form a five, six, or seven-membered ring.

In some embodiments of Formula (1), the organic nitrogen-containing base has the following structure:

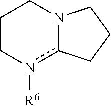

In Formula (1g), R⁶ is as defined earlier above. In some embodiments, R⁶ is a hydrogen atom. In other embodiments, R⁶ is an alkyl group containing one to three carbon atoms, or more particularly, R⁶ is selected from methyl groups, ethyl groups, n-propyl groups, or isopropyl groups or selected from a combination thereof. In the case where R⁶ is a hydrogen atom or alkyl group, the optional double bond is not present. In some embodiments, R⁶ is an electron pair and the nitrogen atom attached to R⁶ is engaged in a carbon-nitrogen double bond.

In some embodiments of Formula (1), the organic nitrogen-containing base has the following structure:

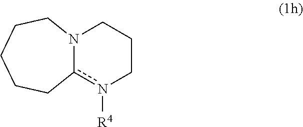

In Formula (1h), R⁴ is as defined earlier above. In some embodiments, R⁴ is a hydrogen atom. In other embodiments, R⁴ is an alkyl group containing one to three carbon atoms, or more particularly, R⁴ is selected from methyl groups, ethyl groups, n-propyl groups, or isopropyl groups or selected from a combination thereof. In the case where R⁴ is a hydrogen atom or alkyl group, the optional double bond is not present. In some embodiments, R⁴ is an electron pair and the nitrogen atom attached to R⁴ is engaged in a carbon-nitrogen double bond.

In some embodiments of Formula (1), the organic nitrogen-containing base has the following structure:

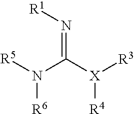

In Formula (1i), R¹, R³, R⁴, R⁵, R⁶ and X are as defined earlier above, except that R¹, R³, R⁴, R⁵, and R⁶ are not permitted to interconnect. In some embodiments of Formula (1i), X is CR^{a}. In other embodiments of Formula (1i), X is N. In some embodiments, all of R¹, R³, R⁴, R⁵, R⁶ are hydrogen atoms. In other embodiments, one, two, three, four, or all of R¹, R³, R⁴, R⁵, R⁶ are alkyl groups containing one to three carbon atoms, or more particularly, one, two, three, four, or all of R¹, R³, R⁴, R⁵, R⁶ are selected from methyl groups, ethyl groups, n-propyl groups, or isopropyl groups or selected from a combination thereof. In some embodiments, R¹, R³, and R⁵ are selected from alkyl groups containing one to three carbon atoms, such as any of the alkyl groups provided above, or R¹, R³, and R⁵ are methyl groups.

Some specific examples of organic nitrogen-containing bases include:

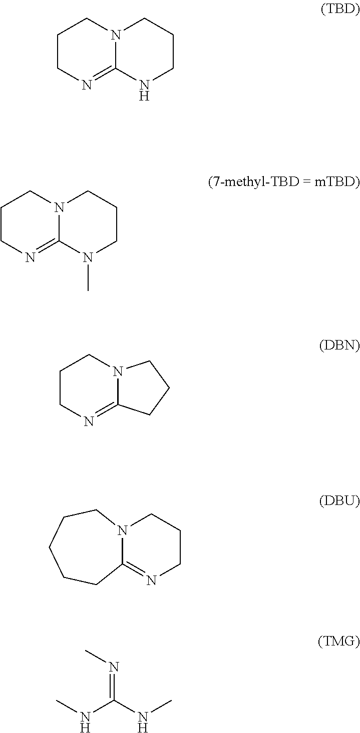

As indicated earlier above, the base, which may have any of the structures within Formulas (1), (1a), (1b), (1c), (1d), (1e), (1f), (1h), (1i), or specific structures provided above, is in combination with (typically, complexed with) a carboxylic acid or ester thereof in the catalyst. The base and acid can be included in the catalyst in any suitable molar ratio, such as any of those provided earlier above. Moreover, any base described above can be combined with any acid described in this disclosure to provide a catalyst.

The carboxylic acid or ester thereof may have the following structure:

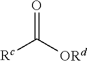

In Formula (2), R^{c} is a hydrogen atom (H) or a hydrocarbon group containing 1-12 carbon atoms, which may be selected from any such hydrocarbon groups (R) described earlier above. In different embodiments, the hydrocarbon group R^{c} contains, for example, 1, 2, 3, 4, 5, 6, 7, 8, 9, 10, 11, or 12 carbon atoms, or a number of carbon atoms within a particular range bounded by any two of the foregoing carbon numbers (e.g., 1-12, 1-8, 1-6, 1-5, 1-4, 1-3, 2-12, 2-8, 2-6, 2-5, 2-4, or 2-3 carbon atoms). In some embodiments, R^{c} is composed solely of carbon and hydrogen. In other embodiments, R^{c} is composed of carbon and possibly hydrogen atoms except that the hydrocarbon group may (i.e., optionally) be substituted with one or more halogen (e.g., fluorine, chlorine, bromine, or iodine) atoms to result in partial or complete halogenation of the hydrocarbon group. The group R^{c} is typically a linear, branched, or cyclic alkyl group or an aromatic (e.g., phenyl) group. Some examples of linear, branched, and cyclic alkyl groups include methyl, ethyl, n-propyl, isopropyl, cyclopropyl, n-butyl, isobutyl, sec-butyl, t-butyl, cyclobutyl, n-pentyl, cyclopentyl, isopentyl, n-hexyl, cyclohexyl, n-heptyl, n-octyl, isooctyl, n-nonyl, n-decyl, n-undecyl, and n-dodecyl groups, as well as fluorinated and chlorinated versions of such groups.

In Formula (2), R^{d} is selected from H and alkyl groups containing 1-3 carbon atoms. In one set of embodiments, R^{d} is a hydrogen atom, and R^{c} may be any of the linear, branched, or cyclic alkyl groups (or a phenyl group) as provided above. In another set of embodiments, R^{d} is an alkyl group, and R^{c} may be any of the linear, branched, or cyclic alkyl groups (or a phenyl group) as provided above.

Some specific examples of carboxylic acids or esters thereof include:

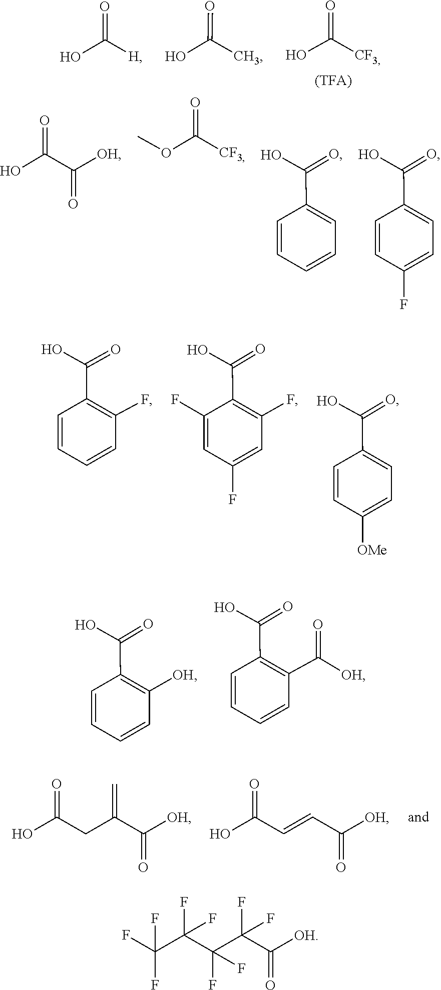

The base may alternatively complex with a sulfonic acid having the general formula R—SO₃R^{e} (where R and R^{e} are as defined above and any specific embodiments thereof), such as:

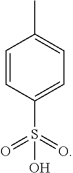

Esters of any of the foregoing acids (i.e., where one or more acidic hydrogen atoms are substituted with one or more R^{d} alkyl groups) are considered herein. Moreover, any base described above may be combined with any acid described above to provide a catalyst. In some embodiments, any base described above, such as TBD or mTBD, may be combined with any of the exemplary acids provided above (e.g., TFA) to result in a catalyst.

The catalyst described above can be produced by combining the base and acid components (i.e., according to Formulas (1) and (2), respectively) under conditions where the two components form a complex with each other. Typically, the two components are combined, in the absence or presence of a solvent, and heated to a temperature of 40-80° C. or more particularly 50-70° C., or about 60° C., for a period of time of 15-45 minutes, more typically about 30 minutes.

The catalyst is typically included in the reaction system in a lower molar amount compared to the molar amount of polymer waste, e.g., no more than 0.25×, 0.2×, 0.1×, or 0.05×, wherein X represents the molar amount of the polymer waste. In some embodiments, after the reaction is complete or near complete, the crude product is mixed with an excess of water to remove excess carbonate molecule and catalyst from breakdown products.

In the method, the R-functionalized carbonate molecule (2) functions as both a reactant and a solvent in the reaction, which eliminates the need of a solvent other than the carbonate molecule (2). Thus, for purposes of the invention, no co-solvent is present in the reaction. Some examples of co-solvents that are excluded from the reaction include alcohols (e.g., methanol and ethanol), acetonitrile, ethers, and acetone. Depending on the R group in the carbonate molecule (2), the carbonate molecule (2) may have a melting point that makes it liquid at room temperature (typically, 20-30° C., or about 25° C.) or it may require heating to an elevated temperature to make it liquid during the reaction. 1f heating is needed to melt the carbonate molecule, the temperature used for this purpose typically corresponds to the elevated temperature used in the reaction for promoting the transesterification reaction.

The number of molar equivalents of carbonate molecule (2) per mole of PET waste (1) is typically at least 1 and up to 5. That is, 1-5 molar equivalents of carbonate molecule (2) is used per molar equivalent of PET waste (1). In different embodiments, precisely or about 1, 2, 3, 4, or 5 molar equivalents of carbonate molecule (2) is used per molar equivalent of PET waste (1). Notably, although a much lower molar ratio of reactant to PET is used compared to the conventional art, the presently described method is capable of producing the terephthalate compound (3) in the substantial absence of ethylene glycol (EG) byproduct. A substantial absence of EG generally refers to no more than or less than 1% EG, or no observable EG. This is a notable achievement since the conventional art typically employs a great excess of the reactant (e.g., an alcohol), such at least 15 or 20 molar ratio of reactant to PET, in an effort to reduce the amount of EG byproduct. Moreover, even with such excess reactant, the conventional art typically produces an appreciable amount of EG byproduct.

In particular embodiments, one or both R groups in the carbonate molecule (2) and the terephthalate compound (3) contains a heterocyclic ring or ring system, which may be selected from any of the saturated or unsaturated heterocyclic rings or ring systems described earlier above. In some embodiments, one or both R groups in the carbonate molecule (2) and the terephthalate compound (3) contains an aromatic ring or ring system. The heterocyclic ring or ring system may be attached to the carbonate oxygen and terephthalate oxygen directly (without a linker) or via a linker, wherein the linker may contain 1-6 carbon atoms and may or may not include one or more heteroatoms selected from N, O, and S. Typically, the heterocyclic ring or ring system attaches via a ring carbon atom and not a ring heteroatom. The resulting terephthalate compound (3) that contains the heterocyclic ring(s) can have a number of uses, including, for example, as ligands for binding to metals, which may aid in the extraction or purification of metals or for producing metal-ligand coordination polymers which are known to have a number of uses (e.g., gas separation and storage).

In particular embodiments, the terephthalate compound (3) has the following structure:

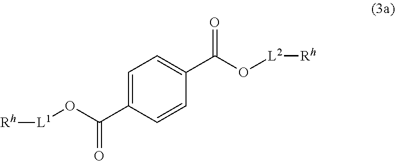

wherein each R^{h} group is a heterocyclic ring or ring system containing at least one ring heteroatom selected from N, O, and S, such as any of the heterocyclic rings or ring systems described in detail above; and L¹ and L² are each selected from a bond and linker containing 1-6 carbon atoms and optionally one or more heteroatoms selected from N, O, and S. In some embodiments, one or both of L¹ and L² is a linear or branched alky linker, such as a linker of the formula —(CH₂)_{g}— wherein g is an integer of 1-6 and one or two H atoms in the linker may be substituted with a methyl group. In separate or further embodiments, one or both of R^{h} is/are selected from N-containing rings (e.g., pyridine), oxygen-containing rings (e.g., furan), or sulfur-containing rings (e.g., thiophene). Notably, any of the heterocyclic rings may contain a substituent, such as a halogen atom, hydroxy group, or ether group.

In some embodiments, one or both R groups in the carbonate molecule (2) and/or the terephthalate compound (3) contain a reactive group. In some embodiments, the reactive group is located at the terminal end of a linear or branched alkyl linkage. The reactive group may be, for example, an activated ester group, such as an N-hydroxysuccinimide (NHS) ester group or derivative thereof, all of which are highly effective in reacting with an amine molecule to form an amide bond. The reactive group may alternatively be, for example, an alkynyl group, which can react with an azide-containing molecule to form a triazole linkage via azide-alkyne click chemistry, as well known in the art. Similarly, the reactive group may be an azide group, which can reactive with an alkynyl-containing molecule to form a triazole linkage via azide-alkyne click chemistry, as well known in the art. As another example, the reactive group may be an epoxide group, which can react with an alcohol, amine, or carboxylic acid compound to form a linkage. As another example, the reactive group may be an isocyanate group, which can react with an alcohol or amine to form a urethane or urea linkage, respectively. As another example, the reactive group may be a maleimide group, which reacts with a thiol by Michael addition to form a thioether bond. Carbonate molecules containing such reactive groups may be reacted with PET to form a terephthalate compound (3) containing such reactive groups, or carbonate molecules containing a precursor of such a reactive group can be reacted with PET to form a terephthalate compound (3) containing a precursor to the reactive group followed by conversion of the precursor reactive group in the terephthalate compound (3) to a reactive group.

In some embodiments, one or both R groups in the carbonate molecule (2) and the terephthalate compound (3) contain a polymerizable group. The polymerizable group may be, for example, an alkenyl group, alkynyl group, azide group, epoxy group, primary alcohol group, primary amine group, isocyanate group, or carboxylic acid or ester group.

In some embodiments, the terephthalate monomer (3), when R is or contains a polymerizable group, is polymerized by subjecting monomer (3) to a polymerization reaction. The polymerization may be achieved by, for example, reaction of monomer (3) with a reactive crosslinking molecule, by copolymerization with another bifunctional monomer, or by self-polymerization. For example, when R in monomer (3) is or includes an alkenyl group, the alkenyl groups may undergo a thiolene polymerization reaction by crosslinking the alkenyl groups in monomer (3) with a dithiol, trithiol, or tetrathiol molecule; or the alkenyl groups in the monomer (3) may undergo self-polymerization by, e.g., vinyl addition or acyclic diene metathesis (ADMET); or the alkenyl groups in the monomer (3) may undergo vinyl addition copolymerization or ADMET copolymerization when the monomer (3) is polymerized in the presence of another alkene-containing monomer, such as styrene, divinylbenzene, or methacrylate. As another example, when R in monomer (3) is or includes an alkynyl group, the alkynyl groups may undergo a thiol-yne “click” polymerization reaction with a dithiol, trithiol, or tetrathiol molecule, or the alkynyl groups may crosslink with a diazide molecule via azide-alkyne click chemistry to form triazole linkages, as well known in the art. As another example, when R in monomer (3) is or includes an azide group, the azide groups may crosslink with a di- or tri-alkyne molecule via azide-alkyne click chemistry to form triazole linkages, as well known in the art. As another example, when R in monomer (3) is or includes an epoxide, the epoxide groups may crosslink with a diol, diamine, or diacid. As another example, when R in monomer (3) is or includes nitrile (CN), the nitrile groups may dimerize or trimerize under suitable conditions, as well known in the art, to form a polymer containing diazine or triazine linkages. As another example, when R in monomer (3) is or includes aldehyde (CHO), the aldehyde groups may crosslink with a diamine molecule to form a polymer containing imine linkages, as well known in the art. As another example, when R in monomer (3) is or includes a terminal amino group (e.g., NHR^{b}, C(O)NHR^{b}, NHC(O)NHR^{b}, or OCONHR^{b}), the terminal amino groups may crosslink with a dicarboxylic acid, diester, or diisocyanate molecule to form a polymer containing amide, urea, or carbamate linkages (where R^{b} is an alkyl group containing 1-3 carbon atoms or a protecting group, such as benzyl, t-butyl, or nitrobenzyl). As another example, when R in monomer (3) is or includes a SR^{b} group, the R^{b} groups may be removed after deconstruction to permit polymerization by disulfide bond formation, copolymerization with di-alkenes, or crosslinking with tri-alkenes. As another example, when R in monomer (3) is or includes a SSR^{b} group, the SSR^{b} groups may polymerize via disulfide bond exchange. As another example, when R in monomer (3) is or includes a B(OH)₂ group, the B(OH)₂ groups may crosslink with a dihalo or ditriflate hydrocarbon molecule by Suzuki-Miyaura cross-coupling, as well known in the art, particularly when the B(OH)₂ groups are bound to an aryl, alkenyl, or alkynyl group also present in R. As another example, when at least one R in monomer (3) is or includes a thiophene ring, the thiophene ring may be polymerized by methods well known in the art to produce a polythiophene backbone.

In some embodiments, the method may further include polymerizing a dialkene monomer according to Formula (3a), wherein both R^{h} groups contain an alkenyl group, by an acyclic diene metathesis (ADMET) polymerization process, wherein the ADMET polymerization process is well known in the art. The ADMET polymerization process is described in, for example, J. T. Patton et al., Macromolecules 25, 3862-3867, 1992, the contents of which are herein incorporated by reference.

The ADMET polymerization process of a dialkene monomer according to Formula (3a) is depicted in the following reaction scheme:

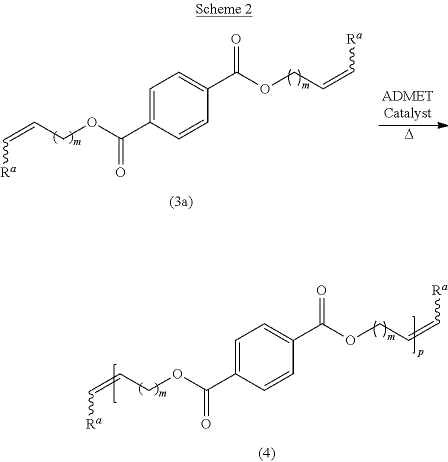

As well known, the ADMET polymerization process employs a Mo-based or Ru-based catalyst under mild elevated temperature conditions (e.g., 40-80° C.) to convert the dialkene monomer to the metathesized polymer. For example, in some embodiments, ADMET polymerization of a dialkene monomer (3a) is achieved by using 2:1 v/v % monomer/solvent in a suitable organic solvent (e.g., 1,2,4-trichlorobenzene) at about 60° C. under dynamic vacuum using G1 catalyst (RuCl₂(═CHPh)(PCy₃)₂) or G2 catalyst (RuCl₂(═CHPh)(H₂IMes)(PCy₃)) with catalyst loadings ranging from 0.2-1 mol %.

In the above Scheme 2, R^{a} is selected from H and alkyl groups containing 1-6 carbon atoms (or more particularly, methyl or ethyl), as described above; m is an integer from 1-6 (or more particularly, 1-4), as described above; and p is at least 10, 20, 30, 40, or 50. The wavy line connecting to R^{a} indicates that R^{a} may be in a cis or trans position. Notably, the ADMET reaction may or may not be conducted in the presence of one or more additional alkene molecules (e.g., styrene, divinylbenzene, or methacrylate) to produce a mixed metathesis product.

In some embodiments, the method further includes subjecting the polymer (4) to a retro-ADMET process in the presence of the hydroxy alkene molecule (2a), ethylene gas, or a mono-functional alkene (e.g., 1-hexene) to afford the dialkene monomer (3a). The retro-ADMET depolymerization process uses the same catalysts and conditions as used for ADMET and is well known in the art. The retro-ADMET process can be used to provide a closed-loop cycle in which ADMET is used to produce the polymer (4) which may be subsequently deconstructed with retro-ADMET to produce a monomer (3a), which can then subsequently be polymerized into the same or other polymer.

A representative retro-ADMET depolymerization process of a polymer (4) is depicted in the following reaction scheme:

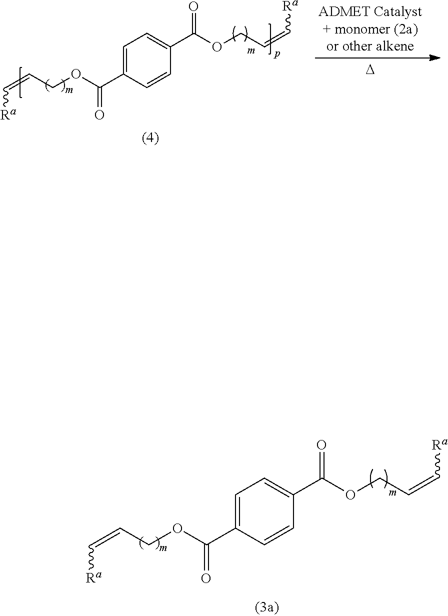

Examples have been set forth below for the purpose of illustration and to describe certain specific embodiments of the invention. However, the scope of this invention is not to be in any way limited by the examples set forth herein.

### Examples

### Synthesis of Model Compounds

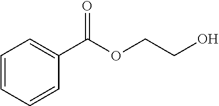 [SMILES: O=C(OCCO)c1ccccc1]

Ethylene glycol monobenzoate (EGMBz). A 100 mL round bottom flask (rbf) equipped with a magnetic stir bar was charged with dichloromethane (DCM, 25 mL), pyridine (2.3 mL, 28 mmol, 1.1 equiv), and ethylene glycol (4.2 mL, 75 mmol, 3 equiv). The rbf was cooled to 0° C. in ice bath and benzoyl chloride (2.9 mL, 25 mmol, 1 equiv) was added to the reactor dropwise. The reaction mixture was gradually warmed to room temperature and allowed to continue stirring overnight. The crude reaction mixture was then transferred to a separatory funnel along with 200 mL ethyl acetate, washed 3× with DI H₂O (50 mL) and brine (50 mL). The organic layer was dried over sodium sulfate and the solvent was removed in vacuo. This was further dried under vacuum to provide the product as a clear, colorless oil (3.1 g, 73%). ¹H NMR (400 MHz, CDCl₃): δ 7.99 (d, 2H), 7.49 (t, 1H), 7.37 (t, 2H), 4.39 (t, 2H), 3.88 (t, 2H). ¹³C NMR (400 MHz, CDCl₃): δ 166.99, 133.20, 129.86, 129.69, 128.43, 66.67, 61.39.

### Representative Polymer Deconstruction Kinetics Protocol

Kinetic experiments were conducted in a similar manner as described in the experimental section. Notable distinctions include carbonate (DMC and DOC) and catalyst identities (m-TBD, TBD, and TBD:TFA) respectively. E ach indicated time point was a separate reaction that was heated for the prescribed time and subsequently removed from heating and allowed to cool to room temperature. Notably, reactions that produced DMT resulted in product crystallization upon cooling. Reaction solutions with crystallized DMT were diluted with CDCl₃ (3 mL) and an aliquot was removed for ¹H NMR analysis to determine soluble aromatics and EC formation/consumption.

Mechanistic Experiments with Model Compounds (EGMBz & EGDBz). A 15 mL thick-walled glass pressure tube was charged with mono or dibenzoate (2.6 mmol), a PTFE stir bar, DMC (5.2 mmol, 2 equiv), and TBD/TFA (0.26 mmol, 0.1 equiv). The Tube was tightly sealed, and the reaction mixture was heated to 180° C. in an oil bath with vigorous stirring. After 2 h, the pressure tube was removed from the heat and allowed to cool to room temperature. The cap was unscrewed, and an aliquot was removed for ¹H NMR analysis.

Mechanistic Experiments with EG as a surrogate for PET. A 100 mL round bottom flask was charged with EG (3.2 mL, 57.38 mmol) DOC (3.3 mL, 11.5 mmol), a PTFE coated stir bar, and TBD (35 mg, 0.25 mmol). The Flask was equipped with a findenser before heating the reaction mixture to 180° C. in an oil bath with vigorous stirring for 24 hours to ensure equilibration and off-gassing of any evolved CO₂. The apparatus was removed from heating and cooled to room temperature and aliquots were removed and diluted to 15 μM in HPLC grade MeOH with respect to DOC and used for HRMS Analysis.

Similar conditions and amounts were employed for the qualitative CO₂ detection experiments; notable differences include the absence of a findenser, using a 2-neck RBF flask so that the reaction headspace could be bubbled into a solution of the desired CO₂ indicator Bromothymol blue or aqueous calcium hydroxide.

An analogous experiment was conducted except DEC was used in place of DOC, a 15 mL pressure tube was used in place of a RBF and the reaction was conducted for 1 h with periodic aliquots removed and residual carbonate was evaporated for 16 h in a vacuum oven prior to ¹H NMR analysis to follow the consumption of EG, the formation of EC, and subsequent polymerization of EC to oligomeric glycolether carbonates.

### Terephthalate Compounds

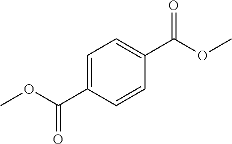

Synthesis of dimethyl terephthalate, DMT, starting from PET pellets. DMT was prepared via the general procedure, using dimethyl carbonate (DMC) as the reaction medium. After venting, the remaining DMC was removed in vacuo and the residue was recrystallized from CH₃OH to afford the desired product DMT as an off-white, crystalline solid (0.45 g, 90% yield). 1H NMR (400 MHz, CDCl₃): δ 8.09 (s, 4H), 3.94 (s, 6H). ¹³C NMR (400 MHz, CDCl₃): δ 166.31, 133.94, 129.58, 52.46.

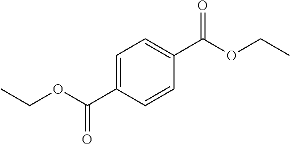 [SMILES: CCOC(=O)c1ccc(C(=O)OCC)cc1]

Synthesis of diethyl terephthalate, DET, starting from PET pellets. DET was prepared via the general procedure, using dimethyl carbonate (DEC) as the reaction medium. After venting the pressure tube, the crude reaction mixture was purified directly by silica gel flash chromatography (10 v/v % EtOAc in hexane) to afford the desired product, DET, as a white, crystalline solid (0.55 g, 95% yield). 1H NMR (400 MHz, CDCl3): δ 8.10 (s, 4H), 4.40 (q, 4H), 1.41 (s, 6H). ¹³C NMR (400 MHz, CDCl₃): δ 165.80, 134.15, 129.44, 61.36, 14.26.

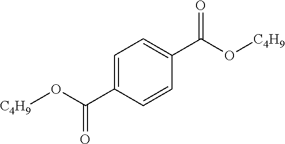

Synthesis of dibutyl terephthalate, DBT, starting from PET pellets. DBT was prepared via the general procedure, using dibutyl carbonate (DBC) as the reaction medium. After venting the pressure tube, the crude reaction mixture was purified directly by silica gel flash chromatography (10 v/v % EtOAc in hexane) to afford the desired product, DBT, as a colorless liquid (0.59 g, 82% yield). 1H NMR (400 MHz, CDCl3): δ 8.09 (s, 4H), 4.35 (t, 4H), 1.76 (p, 4H), 1.48 (m, 4H), 0.98 (t, 6H). ¹³C NMR (400 MHz, CDCl₃): δ 165.95, 134.21, 129.50, 65.31, 30.73, 19.29, 13.79.

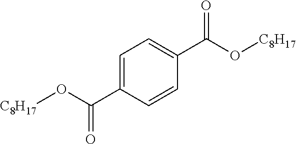

Synthesis of dioctyl terephthalate, DOT, starting from PET pellets. DOT was prepared via the general procedure, using dioctyl carbonate (DOC) as the reaction medium. After venting the pressure tube, the crude reaction mixture was purified directly by silica gel flash chromatography (5 v/v % EtOAc in hexane) to afford the desired product, DOT, as a colorless oil (0.92 g, 91% yield). 1H NMR (400 MHz, CDCl3): δ 8.10 (s, 4H), 4.33 (t, 4H), 1.78 (p, 4H), 1.5-1.2 (m, 20H), 0.88 (t, 6H). ¹³C NMR (400 MHz, CDCl₃): δ 165.97, 134.23, 129.52, 65.64, 31.83, 29.28, 29.23, 28.69, 26.06, 22.69, 14.14.

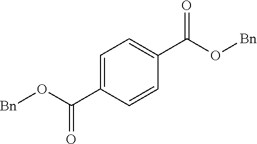 [SMILES: O=C(OCc1ccccc1)c1ccc(C(=O)OCc2ccccc2)cc1]

Synthesis of dibenzyl terephthalate, DBnT, starting from PET pellets. DBnT was prepared via the general procedure, using dibenzyl carbonate (DBnC) as the reaction medium. After venting the pressure tube, the crude reaction mixture was purified directly by silica gel flash chromatography (5 v/v % EtOAc in hexane) to afford the desired product, DBnT, as a colorless, viscous oil (0.79 g, 88% yield). 1H NMR (400 MHz, CDCl3): δ 8.13 (s, 4H), 7.5-7.3 (m, 10H), 5.38 (s, 4H). ¹³C NMR (400 MHz, CDCl₃): δ 165.61, 135.69, 134.01, 129.70, 128.68, 128.44, 128.30, 67.15.

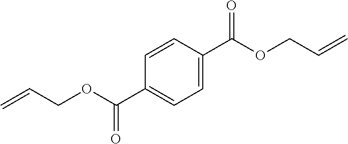 [SMILES: C=CCOC(=O)c1ccc(C(=O)OCC=C)cc1]

Synthesis of diallyl terephthalate, DAT, starting from PET pellets. DAT was prepared via the general procedure, using diallyl carbonate (DAC) as the reaction medium. After venting the pressure tube, the crude reaction mixture was purified directly by silica gel flash chromatography (5 v/v % EtOAc in hexane) to afford the desired product, DBnT, as a colorless liquid (0.45 g, 71% yield). 1H NMR (400 MHz, CDCl3): δ 8.13 (s, 4H), 6.05 (m, 2H), 5.45-5.30 (dd, 4H), 4.85 (m, 4H). ¹³C NMR (400 MHz, CDCl₃): δ 165.49, 134.04, 131.94, 129.66, 118.72, 66.03.

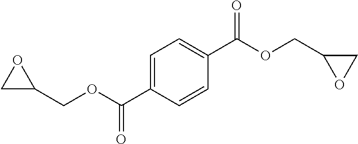 [SMILES: O=C(OCC1CO1)c1ccc(C(=O)OCC2CO2)cc1]

Synthesis of diglycidyl terephthalate (DGT), starting from DAT. A 100 mL RBF was charged with meta-chloro perbenzoic acid (mCPBA, 2.5 g, 77%, 11.1 mmol) a PTFE coated stir bar, and chloroform (CHCl₃, 30 mL). A CHCl₃ (2 mL) solution of DAT (930 mg, 3.77 mmol) was prepared and added dropwise with vigorous stirring. The flask was equipped with a condenser and heated at 40° C. for 72 h. The reaction was cooled to room temperature and then cooled to 5° C. for 16 h. The solids were removed via gravity filtration and solvent was removed in vacuo. The powdery off-white solids were dissolved in DCM (20 mL) and washed with a 1:1 sodium bicarbonate:sodium thiosulfate solution (2×20 mL). The combined organic washes were dried over magnesium sulfate, filtered and solvent removed in vacuo. DGT was isolated as white powder in 70% yield (720 mg, 2.59 mmol). 1H NMR (400 MHz, CDCl3): δ 8.14 (s, 4H), 4.7 (dd, 2H), 4.19 (dd, 2H), 3.36 (m, 2H), 2.92 (t, 2H), 2.74 (dd, 2H). ¹³C NMR (400 MHz, CDCl₃): δ 165.56, 133.84, 129.93, 66.11, 49.48.

Catalysts screened for activity in carbonate interchange deconstruction.

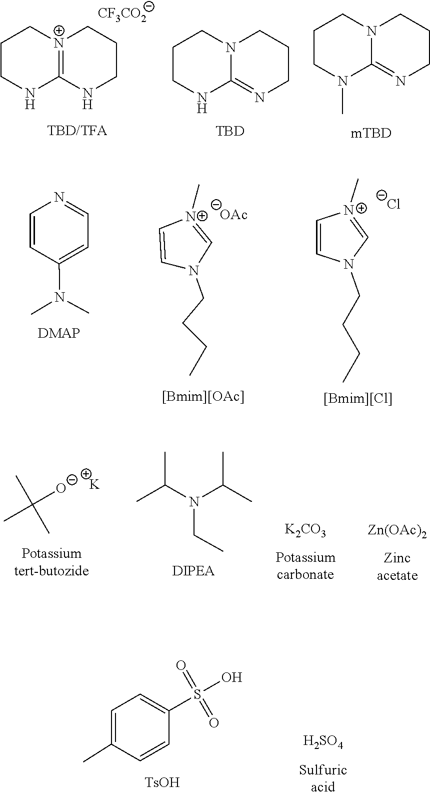

TABLE 1
Conditions used for equilibrium experiments

| | | Equiv. | Vol. Reagent | [TBD/TFA] | Selectivity |
|---|---|---|---|---|---|
| Entry | Reagent | Reagent | (mL) | (mM) | (%)^{a} |
| 1 | Butanol | 20 | 4.8 | 0.055 | 95 |
| 2 | Butanol | 10 | 2.4 | 0.11 | 89 |
| 3 | Butanol | 5 | 1.2 | 0.22 | 81 |
| 4 | Butanol | 3 | 0.71 | 0.36 | 71 |
| 5 | Butanol | 2 | 0.48 | 0.55 | 57 |
| 6 | Octanol | 20 | 8.2 | 0.32 | 97 |
| 7 | Octanol | 10 | 4.1 | 0.64 | 94 |
| 8 | Octanol | 5 | 2.0 | 0.13 | 87 |
| 9 | Octanol | 3 | 1.2 | 0.21 | 77 |
| 10 | Octanol | 2 | 0.82 | 0.32 | 68 |
| 11 | Benzyl | 20 | 5.4 | 0.048 | 96 |
| | alcohol | | | | |
| | (BnOH) | | | | |
| 12 | BnOH | 10 | 2.7 | 0.096 | 90 |
| 13 | BnOH | 5 | 1.4 | 0.19 | 82 |
| 14 | BnOH | 3 | 0.81 | 0.32 | 68 |
| 15 | BnOH | 2 | 0.54 | 0.48 | 58 |
| 16 | Dibutyl | 20 | 9.9 | 0.026 | 100 |
| | carbonate | | | | |
| | (DBC) | | | | |
| 17 | DBC | 10 | 4.9 | 0.053 | 100 |
| 18 | DBC | 5 | 2.5 | 0.11 | 97 |
| 19 | DBC | 3 | 1.5 | 0.18 | 95 |
| 20 | DBC | 2 | 0.99 | 0.26 | 91 |
| 21 | DBC | 1.5 | 0.74 | 0.36 | 89 |
| 22 | DBC | 1 | 0.49 | 0.55 | 80 |
| 23 | Dioctyl | 20 | 17 | 0.016 | 100 |
| | carbonate | | | | |
| | (DOC) | | | | |
| 24 | DOC | 10 | 6.3 | 0.041 | 100 |
| 25 | DOC | 5 | 4.2 | 0.062 | 99 |
| 26 | DOC | 3 | 2.5 | 0.10 | 97 |
| 27 | DOC | 2 | 1.7 | 0.16 | 95 |
| 28 | DOC | 1.5 | 1.2 | 0.21 | 92 |
| 29 | DOC | 1 | 0.83 | 0.31 | 87 |
| 30 | Dibenzyl | 20 | 12.7 | 0.020 | 100 |
| | carbonate | | | | |
| | (DBnC) | | | | |
| 31 | DBnC | 10 | 6.4 | 0.040 | 99 |
| 32 | DBnC | 5 | 3.2 | 0.082 | 99 |
| 33 | DBnC | 3 | 1.9 | 0.14 | 96 |
| 34 | DBnC | 2 | 1.3 | 0.20 | 93 |
| 35 | DBnC | 1.5 | 0.95 | 0.27 | 89 |
| 36 | DBnC | 1 | 0.64 | 0.41 | 81 |
| 31 | Dimethyl | 20 | 4.4 | 0.059 | 100 |
| | carbonate | | | | |
| | (DMC) | | | | |
| 32 | DMC | 10 | 2.2 | 0.12 | 99 |
| 33 | DMC | 5 | 1.1 | 0.24 | 96 |
| 34 | DMC | 3 | 0.66 | 0.40 | 92 |
| 35 | DMC | 2 | 0.44 | 0.59 | 86 |

### Representative Polymer Carbonate-Interchange Deconstruction Protocol

A 15 mL thick-walled glass pressure tube was charged with PET pellets (2.6 mmol, 1 equiv), catalyst (0.01-0.1 equiv), a PTFE coated stir bar, and carbonate (2-20 equiv). The tube was tightly sealed, and the reaction mixture was heated to 180° C. in an oil bath with vigorous stirring. After a prescribed reaction time, the pressure tube was removed from heat and allowed to cool to room temperature. The cap was unscrewed to release the pressure, resulting in significant bubbling. The crude reaction mixture was sampled for analysis by ¹H NMR spectroscopy. The various terephthalate products were then isolated.

### Results and Discussion

### Catalyst Selection for Carbonate-Mediated Deconstruction

Initial experiments sought to identify catalysts to promote PET deconstruction by transesterification and consumption of the evolving EG by irreversible alkylation. The efficacy of various Brønsted catalysts, ionic liquids, and Lewis catalysts, all of which are known catalyze transesterification reactions, were investigated using dimethyl carbonate (DMC) as the reaction medium. In addition to its unique reactivity, DMC is an abundant “green solvent” that can be sourced from biomass or CO₂. In a typical experiment, PET pellets (weight average molecular weight, M_{w}=46 kg mol⁻¹) were suspended in excess DMC (15 wt/v %) in the presence of 10 mol % catalyst and heated at 180° C. in a sealed pressure tube for 2 h. PET conversion was determined by gravimetric analysis and the selectivity of DMT formation was quantified via ¹H NMR spectroscopic analysis of the crude reaction mixtures (see Equations 1 and 2). Quantitative consumption of PET with concomitant formation of DMT with each tested Brønsted base (Table 1, entries 1-4, 7) catalyst except Hunig's base (N,N-diisopropylethylamine) was observed, which can be attributed to its relatively weak basicity and poor nucleophilicity (Table 1, entry 8). The ionic liquid catalysts 1-butyl-3-methyl imidazolium (Bmim) [Bmim][OAc] and [Bmim][Cl] also mediated quantitative conversion of PET to DMT (Table 1, entries 5,6). In contrast, Brønsted and Lewis acid catalysts afforded low conversions of PET and only trace formation of the desired product DMT (Table 1, entries 10-12).

Next, the ¹H NMR spectra were carefully analyzed to determine the impact of catalyst identity on the consumption of the EG byproduct. Although previous studies have indicated that EG can be readily converted to ethylene carbonate, EC, in the presence of DMC, this alone is not sufficient to alter the transesterification equilibrium, as EG can be regenerated from EC via subsequent transcarbonylation reactions. In this work, catalysts were sought that could further convert EC to oligoethers via alkylation, as indicated by the absence of both EG and EC after the deconstruction reaction. It was herein found that superbase catalysts (i.e., 1,5,7-triazabicyclo[4.4.0]decene (TBD) and its derivatives) afforded the lowest yields of EC, which indicates its conversion into poly(ether carbonate) oligomers. As such, TBD and its derivatives TBD/trifluoroacetic acid (TFA) and 7-methyl-triazabicyclodecene (mTBD) were selected for further study (Table 1, entries 1-3).

TABLE 2
Catalyst optimization for CID

| | | | PET | | EC |
|---|---|---|---|---|---|
| | | | Conversion | Selectivity | yield |
| Entry | Catalyst | Classification | (%)^{a} | (%)^{b} | (%)^{c} |
| 1 | TBD:TFA | Guanidine | 100 | >99 | 45 |
| 2 | TBD | superbases | 100 | >99 | 28 |
| 3 | mTBD | | 100 | >99 | 55 |
| 4 | DMAP | | 100 | >99 | 54 |
| 5 | [Bmim][OAc] | Ionic liquids | 100 | >99 | 56 |
| 6 | [Bmim][Cl] | | 100 | >99 | 80 |
| 7 | KOtBu | Other | 100 | >99 | 70 |
| 8 | DIPEA | Brønsted | 1 | 0 | 0 |
| 9 | K₂CO₃ | bases | 1 | 0 | 0 |
| 10 | Zn(OAc)₂ | Lewis acid | 27 | 13 | 0.02 |
| 11 | TsOH | Brønsted | 2 | 0 | 0 |
| 12 | H₂SO₄ | acids | 3 | 0 | 0 |

^{a}PET conversion was determined via gravimetric analysis.
^{b}Product selectivity was determined via ¹H NMR spectroscopic analysis.
^{c}EC yield was determined via ¹H NMR spectroscopic analysis of the crude reaction mixture. These experiments were carried out with 0.5 g of PET suspended in 15 wt/v % of DMC with 10 mol % of the appropriate catalyst at 180° C. for 2 h.

### Transesterification Equilibria

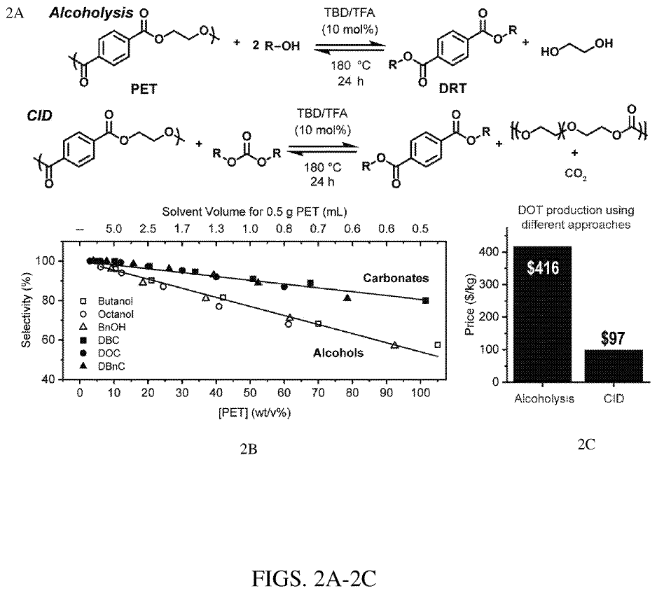

With the optimum catalyst manifold determined, next experiments aimed to compare the equilibrium behavior of PET deconstruction in the presence of various alcohols or carbonates selected to enable direct comparisons. A series of experiments were conducted using different volumes of alcohol or carbonate reagent as the reaction medium at 180° C. for 24 h to ensure equilibration (see schemes in FIG. 2A). TBD/TFA (10 mol %) was used as the catalyst for these experiments due to its excellent thermal stability over long reaction times. Notably, PET was fully consumed in each trial as judged gravimetrically, even with just 1 molar equiv. of added reagent. After completion, an aliquot of each reaction mixture was removed and diluted in CDCl₃ for ¹H NMR spectroscopic analysis. Product selectivity, in terms of the percentage of terephthalate esters derived from the alcohol or carbonate, was determined via relative integration of the aromatic signals to those of the methylene protons adjacent to the terephthalate ester motifs. As shown in FIG. 2B, the selectivity of transesterification when using alcohols decreased rapidly with decreasing reagent volume (increasing PET concentration). In stark contrast, deconstructions mediated by carbonates were far less sensitive to stoichiometry and maintained good selectivity even at high PET concentration. Selectivities of 80%, 87%, and 81% were achieved when using just 1 molar equiv. of dibutyl carbonate (DBC), dioctyl carbonate (DOC), or dibenzyl carbonate (DBnC), respectively, compared to just 58%, 68%, and 57% for butanol, octanol, and benzyl alcohol (BnOH).

To further illustrate the advantage of the present methodology, a preliminary technoeconomic analysis (TEA) of CID compared to alcoholysis was conducted based on the conditions used for determining product equilibrium. DOC and 1-octanol were selected as the substrates of choice for this analysis. To achieve ~99% product selectivity and thus molar conversion at 0.5 g scale of PET, a vast excess of 1-octanol (50 mL) was required in contrast to the carbonate congener (4.2 mL). As shown by the results in FIG. 2C, this significant solvent excess in the case of alcoholysis was needed to combat the alcoholysis equilibria and resulted in a stark 4× increase in material costs for alcoholysis as compared with CID. Taken together, these data demonstrate that carbonates provide a significant advantage for PET deconstruction by transesterification as compared to alcohols. As shown, the carbonates provide good selectivity with minimal to no stoichiometric excess.

### Mechanistic Considerations

Next experiments were aimed at better understanding the mechanistic underpinnings of the observed high selectivity of CID using model experiments. Ethylene glycol monobenzoate (EGBz) and octyl benzoate (OBz) were subjected to 2 molar equiv. of DMC in the presence of 10 mol % TBD/TFA at 180° C. for 1 h. Methyl benzoate (MBz) was produced with 86% selectivity when starting from EGBz, while a lower selectivity of 58% was obtained from OBz. The key difference between these two experiments was the presence or absence of EG, which drove the transesterification equilibrium via its conversion to oligoethers. Small molecule PET surrogates were further employed to interrogate the involvement of PET hydroxyl end groups in the deconstruction mechanism. Ethylene glycol dibenzoate (EGDBz) and EGBz were treated with 5 molar equiv. of DMC and 10 mol % TBD/TFA at 180° C. for 1 h. Quantitative conversion to MBz was observed for both PET analogues. These results implicated acyl transfer reactions, mediated by the catalyst, in generating an initial fraction of nucleophilic alcoholic species required for transesterification (see, for example, R. C. Pratt et al., J Am Chem Soc. 2006; 128(14):4556-7).

Kinetic monitoring was employed to follow the time-dependent consumption of PET relative to the formation of the key byproduct EC (from EG). Experiments were carried out in parallel using 10 mol % of either TBD, TBD/TFA, or mTBD and 5 molar equiv. of DMC at 180° C. and stopped at specified time points by removing the reaction vessels from heat. Aliquots of these mixtures were subjected to ¹H NMR spectroscopic analysis to track the formation of EC, while PET conversion was determined via gravimetric analysis on the recovered PET solids. Kinetic studies using TBD/TFA or mTBD exhibited notable induction periods during the first 10 min, with nearly quantitative PET mass recovery at these timepoints. PET conversion increased rapidly after these induction periods, accompanied by increased evolution of EC. PET was completely consumed by 25 min and EC yield increased up to 0.55 molar equiv. relative to the terephthalate product. Prolonged reaction times affected a significant decrease in the quantity of EC, to <0.2 molar equiv., consistent with its consumption via polymerization. Extending this kinetic investigation to TBD revealed similar trends, but on a much faster time scale and with no apparent induction period. In this system, complete PET consumption was observed within 10 min concomitant with EC formation and consumption.

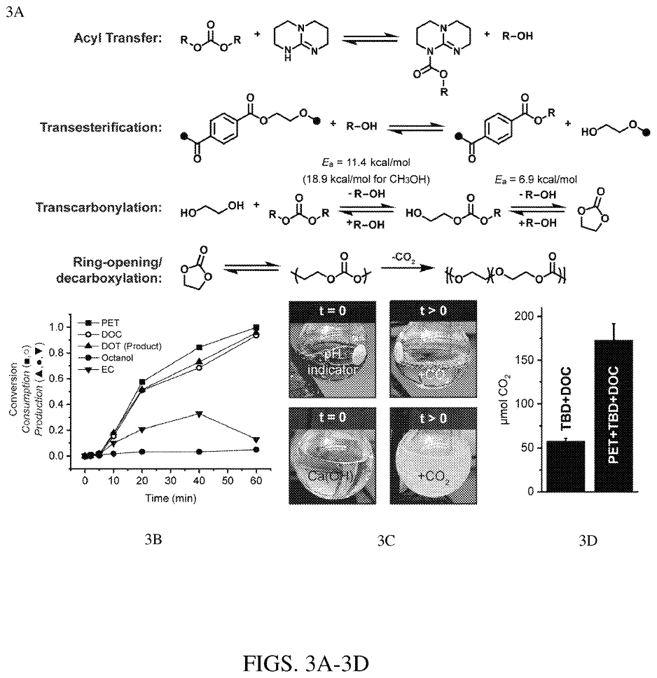

A more in-depth kinetic analysis was conducted to monitor the consumption and formation of all key species. The reactions performed for the kinetic analysis are schematically shown in FIG. 3A. Dioctylcarbonate (DOC) was used for this experiment due to the low volatility of DOC itself and its constituent alcohol, 1-octanol. This factor permitted its terminal methyl motifs to be used as an internal standard. TBD was used as the catalyst based on its relatively higher activity compared with TBD/TFA or mTBD. As before, each time point was represented by an individual reaction, with conversion values calculated from a combination of ¹H NMR spectroscopic and gravimetric analyses. As shown in FIG. 3B, DOT formation was commensurate with the consumption of both PET and DOC. Approximately 1 of the 5 molar equiv. of DOC was consumed (i.e., 20%), suggesting that each equiv. of carbonate supplied the necessary 2 equiv. of alcohol to form the diester product. These processes occurred in tandem with EC formation and consumption, with EC concentration reaching a maximum at ca. 40 min before decreasing. Notably, the concentration of 1-octanol, derived from reaction with DOC, remained consistently low throughout the course of the reaction and was on the same order as the loading of TBD.

CID involves reactions between alcohols and carbonates via several distinct pathways. These pathways are shown in FIG. 3A. Briefly, EG released from PET or alcohol from the carbonate can both participate in intermolecular carbonylation (B_{AC}2) or methylation (B_{AL}2) reactions, while the asymmetric carbonate product can undergo an additional intramolecular carbonylation reaction to form EC. DFT computed activation energies for these various pathways revealed a slight kinetic preference for B_{AC}2 reactions involving EG, with the intramolecular reaction exhibiting the lowest barrier.

To confirm the liberation of CO₂ during CID, qualitative experiments were first performed utilizing bromothymol blue, a colorimetric indicator that is frequently used to detect the presence of carbonic acid. The reaction headspace of a PET deconstruction reaction was bubbled in the presence of DOC and TBD into a bromothymol blue solution. After 8 min, the solution turned bright yellow, which indicates the evolution of CO₂ (see FIG. 3C). Further confirmation was provided via bubbling the reaction atmosphere into a limewater solution (CaOH in H₂O), which resulted in the precipitation of insoluble CaCO₃. Following these qualitative experiments, next experiments were aimed at quantifying the amount of CO₂ released throughout the deconstruction. Analogous deconstruction reactions were conducted, and the headspace was analyzed via GC-MS, which indicated ca. 180 μmol of CO₂ was present after 2 h, >3× higher than was observed for a comparative reaction conducted with no added PET (see FIG. 3D).

To assess EC polymerization, additional reactions were conducted in which EG was used in place of PET. EG was almost fully converted to EC (>95%) within 2 min when reacted with 5 molar equiv. of diethyl carbonate (DEC) and 1 mol % TBD at 180° C. The generated EC was then consumed in pseudo-first-order fashion over the next 58 min, thereby resulting in the formation of various carbonate and ether species as determined by ¹H NMR spectroscopy. EC consumption was accompanied by a gradual shift in the ratio of ether and carbonate products, which is consistent with the conversion of carbonates to ethers via decarboxylation. High resolution mass spectrometry (HRMS) analysis of a similar reaction between EG and DOC that was left open to air at 180° C. for 24 h to promote CO₂ off-gassing confirmed the formation of various glycol ether products, including tetraethylene and hexaethylene glycol, diethylene glycol monooctyl ether, and 18-crown-6 ether.

Based on the findings from model reactions and kinetic experiments, and considering the reported reactivity of carbonates in the chemical literature, the following conclusions regarding the mechanism of CID were drawn: (1) the catalyst (i.e., TBD) was responsible for the generation of free nucleophile via acyl transfer, and the concentration of free nucleophile was proportional to catalyst loading throughout the course of the reactions; (2) both acyl transfer and proton transfer catalysis were in operation simultaneously, with the former providing faster overall reaction kinetics; (3) EG generated by transesterification with PET was rapidly converted to EC via reaction with carbonate and subsequent intramolecular cyclization; and (4) EC was polymerized in the later stages of the reaction to form poly(carbonate)s, which were gradually converted to polyethers via transalkylcarbonylation coincident with the generation of CO₂. The irreversibility of this final process drove the selectivity of the CID reactions, ultimately suppressing glycolated byproduct formation via the removal of EG from the reaction equilibrium.

### Carbonate Scope Exploration

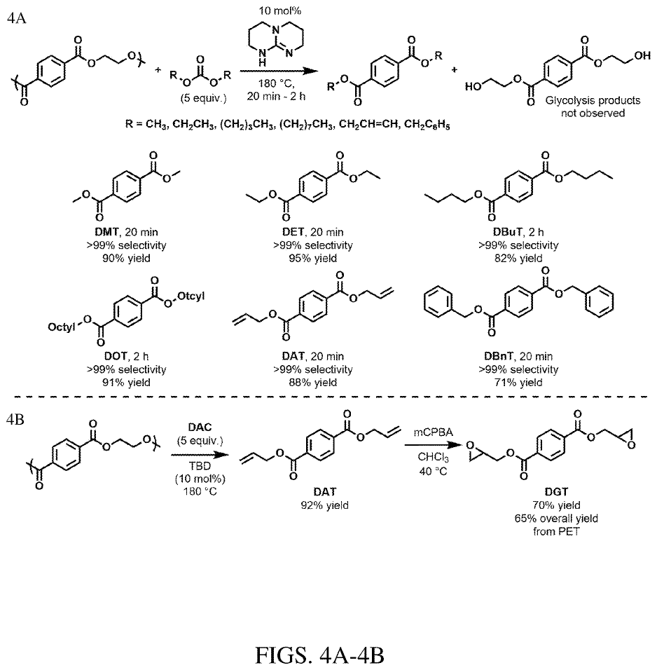

Based on the success of PET deconstruction using carbonates, next experiments tested a library of valuable terephthalate products that could be accessed from plastic waste by employing carbonates with diverse functionality. Symmetrical carbonates such as diethyl, dibutyl, dioctyl, diallyl and dibenzyl carbonate were selected due to their liquid form, commercial availability, and value of the resulting terephthalate product. Notably, these carbonates can be prepared sustainably via CO₂ and the appropriate alcohol (e.g., J. Mutlu et al., Green Chem. 2012; 14(6):1728-35 and W. S. Putro et al., ChemSusChem. 2021; 14(3):842-6). With these targets in mind, next experiments assessed if the PET deconstruction selectivity achieved using these carbonates would be comparable to our initial experiments. A series of PET deconstructions were conducted using fixed reaction stoichiometry and temperature and varying the carbonate used (10 mol % TBD, PET pellets suspended in 5 molar equiv. of the respective carbonate, 180° C.). Quantitative conversion of PET and concomitant formation of DMT, diethyl terephthalate (DET), diallyl terephthalate (DAT), or dibenzyl terephthalate (DBnT) as the sole terephthalate product was observed within 20 min when their corresponding carbonates were used see (FIG. 4A). While similar conversions and yields were observed with dibutyl terephthalate (DBC) or dioctyl terephthalate (DOC), longer deconstruction times (2 h) were required. The resulting terephthalate products could be easily isolated from the deconstruction reaction mixtures via recrystallization or chromatography, thereby affording good isolated yields.

With the library of terephthalate products in hand, next experimenting were aimed at demonstrating how their embedded reactive motifs could be further elaborated to access other high value terephthalates that cannot be prepared directly from PET due to various side reactions at elevated temperature. Notably, some reactive functional groups, such as epoxides, are incompatible with the conditions of PET deconstruction. In the present work, a simple two-step pathway was developed to synthesize diglycidyl terephalate (DGT) via the functional intermediate DAT. In a typical experiment, a transparent PET egg carton was cut into rectangular slices and deconstructed to DAT using standard conditions. DAT was isolated in 92% yield from the crude reaction mixture via flash column chromatography. As shown schematically in FIG. 4B, DAT from PET waste was then subjected to mild epoxidation conditions with excess meta-chloroperbenzoic acid (m-CPBA) at 40° C., after which DGT was isolated as a white powder in 70% yield, or in 65% overall yield starting from PET.

### Demonstration Using Mixed PET Waste

To demonstrate the deployability of this methodology and its tolerance of the various impurities present in post-consumer plastic waste, other experiments employed a deconstruction reaction using a mixture of pristine PET (3.25 g), a PET water bottle (0.69 g of chopped pieces), and a PET egg carton (0.54 g of chopped pieces). This mixture was suspended in DMC (5 molar equiv.) in a sealed pressure tube and heated to 180° C. in the presence of 1 mol % TBD for 3.5 h. After cooling to room temperature, the unreacted DMC was recovered via distillation (79% of the total mass used), and the resulting solid residue was recrystallized from MeOH to afford DMT in good purity and yield (89%) as confirmed by ¹H NMR and gas-chromatography mass spectrometry analysis. This selective deconstruction eliminated the need for upfront separation and cleaning of the mixed plastic waste and enabled ca. 4.5 g of PET from various sources to be directly converted to DMT using just 9.8 mL of carbonate and 0.034 g of catalyst.

Other experiments attempted CID starting from a mixed plastic waste feedstock (FIG. 5B). Pristine PET (4.12 g), a PET egg carton (1.01 g), a PE grocery bag (0.36 g), and a PP cap from a soda bottle (1.74 g) were suspended in 5 molar equiv. of DMC and 1 mol % TBD. After heating in a sealed pressure tube at 180° C. for 4 h, the DMT product was easily separated from the other plastic impurities via a simple 4-step process involving dilution with tetrahydrofuran, filtration, concentration, and recrystallization from MeOH. As above, DMT was obtained in high isolated yield (92%) and good purity and minimal DMC (11.2 mL) and TBD (0.037 g) were used.

### CONCLUSION

In summary, the present work has demonstrated the successful upcycling of PET waste by CID in the presence of nucleophilic catalysts to afford valuable organic terephthalates. This methodology permits precise control over transesterification equilibria, thereby favoring the formation of a single terephthalate product over glycolated byproducts as confirmed by DFT calculations and model and kinetic experiments. A library of carbonates was demonstrated to be amenable to this strategy, thereby affording a variety of useful organic compounds with unique or novel architectures inaccessible by direct alcoholysis. CID was also extended to post-consumer mixed plastic waste, thus demonstrating the resiliency of this method to the various impurities and contaminants commonly encountered therein. The present results highlight the many advantages of using carbonates over traditional alcohols in the deconstruction of condensation polymers, including high selectivity, good yields and low solvent loading. This technology offers a robust foundation for the development of novel deconstruction products derived from various condensation polymer waste streams, and thus, this technology can facilitate a circular carbonate economy in which CO₂ captured from the environment and other various processes can be converted to symmetric carbonate solvents for use in PET waste deconstruction.

While there have been shown and described what are at present considered the preferred embodiments of the invention, those skilled in the art may make various changes and modifications which remain within the scope of the invention defined by the appended claims.

What is claimed is:

1. A method of deconstructing and upcycling polyethylene terephthalate (PET) waste into at least one useful breakdown product, the method comprising contacting the PET waste (1) with a catalyst that facilitates a transesterification reaction in the presence of an R-functionalized carbonate molecule (2) under transesterification conditions to produce a terephthalate monomer (3), according to the following reaction scheme: 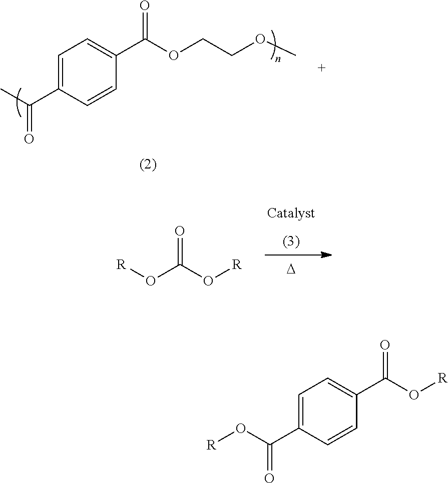
wherein:
R is a hydrocarbon group containing 1-30 carbon atoms and optionally containing one or more heteroatoms selected from N, O, S, P, B, Si, and halogen atoms, wherein the two R groups in molecule (2) may be the same or different and the two R groups in molecule (3) may be the same or different;
n is at least 10; and
the symbol Δ denotes an elevated temperature sufficient to promote a transesterification reaction between (1) and (2) in the presence of the catalyst;
provided that the R-functionalized carbonate molecule (2) functions as both a reactant and a solvent in the reaction, and no co-solvent is present in the reaction.

2. The method of claim 1, wherein the catalyst comprises an organic nitrogen-containing base and a carboxylic acid or ester thereof.

3. The method of claim 2, wherein the organic nitrogen-containing base is a bicyclic guanidine having the following structure: 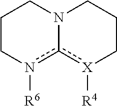
wherein R⁴ and R⁶ are selected from H and CH₃ groups, and the dotted lines represent optional double bonds.

4. The method of claim 3, wherein the bicyclic guanidine is triazabicyclodecene (TBD) or 7-methyl-TBD.

5. The method of claim 2, wherein the catalyst comprises triazabicyclodecene (TBD) or 7-methyl-TBD and a carboxylic acid.

6. The method of claim 5, wherein the carboxylic acid is trifluoroacetic acid (TFA).

7. The method of claim 1, wherein the catalyst comprises 1:1 TBD:TFA.

8. The method of claim 1, wherein the elevated temperature is within a range of 50-200° C.

9. The method of claim 1, wherein the elevated temperature is within a range of 50-180° C.

10. The method of claim 1, wherein the elevated temperature is within a range of 100-200° C.

11. The method of claim 1, wherein the elevated temperature is within a range of 100-180° C.

12. The method of claim 1, wherein 1-5 molar equivalents of R-functionalized carbonate molecule (2) is used per molar equivalent of PET waste (1).

13. The method of claim 1, wherein the PET waste (1) is completely converted to terephthalate monomer (3) within a period of no more than 6 hours.

14. The method of claim 1, wherein ethylene glycol byproduct is not produced in the reaction.

15. The method of claim 1, wherein R is a hydrocarbon group containing 12-30 carbon atoms.

16. The method of claim 1, wherein R is a linear or branched alkyl or alkenyl group containing 1-30 carbon atoms.

17. The method of claim 16, wherein R is a linear or branched alkyl or alkenyl group containing 12-30 carbon atoms.

18. The method of claim 1, wherein R is a linear or branched alkyl group terminated by a polymerizable or reactive group.

19. The method of claim 1, wherein at least one R is a heterocyclic ring or ring system, wherein the heterocyclic ring or ring system contains at least one ring heteroatom selected from N, O, and S, and the compound is non-polymeric.

20. A non-polymeric compound having the following structure: 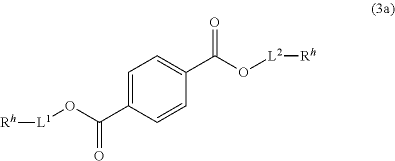
wherein:
each R^{h} group is a heterocyclic ring or ring system, wherein the heterocyclic ring or ring system contains at least one ring heteroatom selected from N, O, and S; and
L¹ and L² are bonds or linkers containing 1-6 carbon atoms and optionally one or more heteroatoms selected from N, O, and S.

21. The compound of claim 20, wherein each R^{h} is a pyridine ring.

22. The compound of claim 20, wherein each R^{h} is a thiophene ring.
